SEQUENCE LISTING (5 sequences)

SEQ ID NO: 1 - protein, 98 aa
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 2 - protein, 12 aa
EPKSCDKTHTCP

SEQ ID NO: 3 - protein, 112 aa
PCPAPELLGGPSVFLFPPKPKDTLMISRTPEVTCVVVDVSHEDPEVKFNWYDGVEVHNAKTKPREEQYNSTYRVVSVLTVLHQDWLNGKEYKCKVSNKALPAPIEKTISKAK

SEQ ID NO: 4 - protein, 107 aa
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 154 aa
AAGSITTLPALPEDGGSGAFPPGHFKDPKRLYCKNGGFFLRIHPDGRVDGVREKSDPHIKLQLQAEERGVVSIKGVCANRYLAMKEDGRLLASKCVTDECFFFERLESNNYNTYRSRKYTSWYVALKRTGQYKLGSKTGPGQKAILFLPMSAKS

(19) United States
(12) Patent Application Publication
(10) Pub. No.: US 2004/0102369 A1
(43) Pub. Date: May 27, 2004

(54) Transport of basic fibroblast growth factor across the blood brain barrier

(75) Inventors: Dafang Wu, Westland, MI (US); William M. Pardridge, Pacific Palisades, CA (US)

Correspondence Address: David J. Oldenkamp, Esq., SHAPIRO & DUPONT LLP, Suite 700, 233 Wilshire Boulevard, Santa Monica, CA, 90401, US

(73) Assignee: The Regents of the University of California

(21) Appl. No.: 10/307,165

(22) Filed: Nov. 27, 2002

(51) Int. Cl. A61K 38/18

(52) U.S. Cl. 514/12

(57) ABSTRACT

Compositions and methods for increasing the receptor mediated transport of basic fibroblast growth factor (bFGF) across the blood brain barrier (BBB). The bFGF is conjugated to a BBB targeting agent using either avidin-biotin technology or genetic engineering. The bFGF conjugate was found to cross the BBB at substantially increased rates while still retaining biological activity. In addition, uptake of the bFGF conjugate by non-brain tissue and organs was limited. The bFGF conjugate may be injected intravenously to provide neuroprotection in patients suffering from cerebral stroke.

### BACKGROUND OF THE INVENTION

1. Field of the Invention

The present invention relates generally to the use of basic fibroblast growth factor (bFGF) to treat disorders of the brain and central nervous system. More particularly, the invention is directed to increasing the ability of bFGF to cross the blood brain barrier (BBB) so that it can be used as an effective neuroprotective agent for treating ischemic stroke and other disorders of the brain.

2. Description of Related Art

The publications and other reference materials referred to herein to describe the background of the invention and to provide additional detail regarding its practice are hereby incorporated by reference. For convenience, the reference materials are identified by author and date and grouped in the appended bibliography.

Ischemic stroke affects more than 500,000 patients a year in this country and millions of people a year in the world. Approximately 80% of the strokes are caused by arterial occlusions secondary to either thrombosis or embolism. Currently, patients with acute ischemic stroke may be only treated with thrombolytic agents. However, clinical efficacy of the thrombolytic agents is limited because these agents (a) can cause brain hemorrhage, and (b) these agents provide no neuroprotection of brain cells during the stroke attack. Whereas thrombolytic agents are limited to reduction of thrombus formation in the vasculature, neuroprotective agents actually work within the brain to limit the death and promote the survival of brain cells during a stroke. There presently are no neuroprotective agents currently available for the treatment of acute stroke. Owing to the lack of effective therapies for ischemic stroke, research interest in neuroprotective agents has been increasing.

Fibroblast growth factors (FGF) are a group of structurally related polypeptides that stimulate various biological functions of fibroblasts, epithelial cells, neuronal cells and smooth muscle cells. There are at least eighteen different fibroblast growth factors (FGF-1 to FGF-18) that range in size from 15 to 23 kilodaltons. One of the more extensively investigated fibroblast growth factors is FGF-2, which is also known as basic fibroblast growth factor (bFGF), heparin-binding growth factor 2 (hbgf-2) or prostatropin.

Basic FGF has been used as a potent angiogenic agent for treating coronary artery disease (see U.S. Pat. No. 6,440,934). Basic FGF has also been shown to have neuroprotective effects in a variety of pre-clinical studies. (Hakan et al., 1999). Basic FGF was found to protect cultured neurons in vitro against various insults, such as hypoglycemia, anoxia, excitatory amino acids and ethanol (Matton and Barger, 1995; Luo et al., 1997). The underlying mechanism of bFGF neuroprotection may be multifactorial, including down-regulation of excitatory amino acid receptor function (Brandoli et al., 1988, Guo et al., 1999) or increased glutamate transport (Casper and Blum, 1995), activation of a mitogen protein kinase (Abe and Saito, 2000), and promotion of neuronal circuit formation (Nakagami et al., 1997).

In vivo, intra-cisternal injection of bFGF reduces infarction volume (Koketsu et al., 1994) and prevents retrograde neuronal death in the thalamus (Yamada et al., 1 991) in a focal cerebral ischemia model in rats. In addition, bFGF was found to be neuroprotective in cerebral ischemia following intracerebroventricular (i.c.v.) injection (Lyons et al., 1991).

The bFGF was administered in the above studies by i.c.v. injection because prior work had shown that bFGF does not cross the blood-brain barrier (BBB) in pharmacologically significant amounts (Whalen et al., 1989). In the absence of BBB disruption, the intravenous administration of bFGF does not cause neuroprotection in focal brain ischemia using the middle cerebral artery occlusion (MCAO) model (Roberts et al., 1995; Harukum et al, 1998). If BBB disruption is present in experimental brain ischemia, then bFGF was found to be neuroprotective following the intravenous administration of high doses (135 μg/kg) in rats subjected to the MCAO model (Fisher et al., 1995; Ay et al., 1999). However, clinical trials of bFGF in human subjects show that such high doses of bFGF cause undesirable side effects (Clark et al., 2000; Lahman et al., 2000). The administration of high doses of bFGF is required due to the modest rate of bFGF penetration into the brain from blood across the BBB. The BBB transport of ¹²⁵I-bFGF is relatively slow and occurs via absorptive-mediated transcytosis of this cationic peptide (Deguchi et al., 2000).

There is a present need to find an effective way to deliver biologically active bFGF to the brain. Intravenous administration is a preferred route of introducing bFGF to the brain. However, this is not possible because the intravenous dosage levels required to achieve a neuroprotective effect in the brain are so high that they are toxic. Direct delivery to the brain using i.c.v. or other BBB disruptive techniques is also undesirable. Accordingly, new compositions and methods are needed where bFGF is somehow modified or otherwise re-formulated to increase transport of biologically active bFGF from the blood stream across the BBB and into the brain.

### SUMMARY OF THE INVENTION

The present invention involves the discovery that bFGF can be conjugated to a suitable transport vector or “molecular Trojan horse” using the avidin-biotin linkage system to form a conjugated composition that is capable of undergoing receptor mediated transcytosis across the blood brain barrier. It was further discovered that the bFGF conjugate not only crosses the BBB in significant amounts, but that once inside the brain, the bFGF conjugate is an effective neuroprotective agent that is capable of reducing the size of cerebral infarctions. In addition, the bFGF conjugate was found to be selectively targeted to the brain in preference over other tissues or organs in the body. The unexpected observation was made that the bFGF conjugate is the most potent intravenous neuroprotective agent discovered to date and is 500% more potent than other neurotrophin conjugates.

The invention covers compositions that include bFGF conjugated to a BBB targeting agent (TA), and there are multiple approaches for attaching the non-transportable drug (bFGF) to the molecular Trojan horse or TA. In one approach, called the avidin-biotin method, biotinylated bFGF (bio-bFGF) is conjugated to a transport vehicle that is made up of a BBB TA and avidin or streptavidin (SA). The conjugate of the TA and SA is designated TA-SA, and the conjugate of the TA and avidin is designated TA-avidin. The TA-SA or TA-avidin complexes may be prepared with either chemical coupling methods or genetic engineering as described in U.S. Pat. No. 6,287,792. In the genetic engineering approach, the gene encoding avidin or SA is fused to the region of the TA gene corresponding to either the amino or carboxyl terminus of the TA protein. The final composition is formed by separately preparing the bio-bFGF and the TA-SA (or TA-avidin) and then mixing the 2 vials just prior to administration to form the bio-bFGF/TA-SA complex, or bio-bFGF/TA-avidin. Owing to the very high affinity of SA or avidin binding of biotin, the bio-bFGF/TA-SA or bio-bFGF/TA-avidin complex is formed immediately after mixing the bio-bFGF and the TA-SA or TA-avidin.

The conjugation of bFGF and the BBB targeting agent using the avidin-biotin bond does not adversely affect the biological properties of the bFGF after it undergoes receptor mediated transcytosis across the BBB. This is because the bFGF in the form of the bio-bFGF/TA-SA complex still binds the bFGF receptor. The retention of the biological activity of the bFGF following biotinylation and conjugation to TA-SA was unexpected, since prior work had shown that when certain neurotrophins, such as epidermal growth factor (EGF) are biotinylated and conjugated to the TA-SA, the EGF neurotrophin would no longer bind to its cognate receptor, which was the EGF receptor (Deguchi et al, 1999). A second general method for attachment of the bFGF to the TA is the genetic engineering method. In this approach, the gene encoding for bFGF is fused to the region of the TA gene corresponding to the amino or carboxyl terminus of the TA protein. FGF fusion genes and biologically active fusion proteins have been genetically engineered and expressed (McDonald et al, 1996; Dikov et al, 1998).

Although any number of BBB targeting agents may be conjugated to bFGF, the present invention is particularly well suited for delivering bFGF to the human brain. The preferred BBB targeting agent binds to the human insulin receptor. In addition, even though any number of brain conditions may be treated using the present bFGF compositions, the preferred use is as a nueroprotective agent for treating cerebral stroke. The amount of bFGF that must be administered intravenously to produce a neuroprotective effect is significantly reduced when the bFGF is conjugated to a BBB targeting agent in accordance with the present invention. This reduction in dosage amount is particularly important in view of the established toxicity of bFGF. With this invention, the systemic dose of bFGF that is administered is reduced by at least a log order of magnitude, which allows for neuroprotection in brain with minimal uptake in non-brain organs.

The above discussed and many other features and attendant advantages of the present invention will become better understood by reference to the detailed description when taken in conjunction with the accompanying drawings.

### BRIEF DESCRIPTION OF THE DRAWINGS

FIG. 1 depicts competition curves of bFGF and its analogues in the radio-receptor binding assay using baby hamster kidney derived cells (BHK-21) in tissue culture and [¹²⁵1]-bFGF as the tracer. Each point on the graph represents the mean of triplicate incubations.

FIG. 2A depicts the plasma profile of unconjugated [¹²⁵I]-bFGF conjugated to the OX26/-SA vector (closed circles) after an IV injection in the rat. FIG. 2B shows the time course of the % of plasma radioactivity that is precipitable with trichloracetic acid (TCA) after intravenous (IV) injection of either the unconjugated or conjugated [¹²⁵I]-bFGF. Each point of the graph represents the mean ±S.E. of three rats.

FIG. 3 shows organ uptake, expressed as percentage of injected dose (ID) per gram organ (% ID/g) of either unconjugated [¹²⁵I]-bFGF or [¹²⁵I]-bFGF conjugated to the OX26-SA vector following IV administration in the rat. Data are mean ±S.E. of three rats. OX26 is a monoclonal antibody against the rat transferrin receptor (Jefferies et al, 1984); this antibody crosses the blood-brain barrier via the transferrin receptor and acts as the targeting agent for bFGF delivery to rat brain.

FIG. 4 shows time course of brain uptake, expressed as the brain volume of distribution (Vd) of [¹²⁵I]-bio-bFGF either unconjugated or conjugated to the OX26-SA vector following internal carotid artery perfusion in the rat. Each point on the graph is the mean ±S.E. of three rats. The brain Vd of [¹⁴C]-sucrose, a marker of brain vascular volume, is also shown.

FIG. 5 shows the neuroprotective effect of bFGF analogs in the mixed rat forebrain cortical cell cultures subjected to hypoxia (24 h)/reoxygenation (4 h) in the 3-(4,5-dimethylthiazol-2-yl-2,5-diphenyltetrazolium bromide (MTT) assay. Each bar in the graph represents mean ±S.D. of triplicate incubations. Each peptide or conjugate was tested in three graded doses and was compared with the nontreated incubations. *, p<0.05; **, p<0.01; ***, p<0.001.

FIG. 6 shows the infarct-reducing effect in a middle cerebral artery occlusion (MCAO) rat model. The first group received vehicle (1.2 ml/kg of buffer containing 1% bovine serum albumin (BSA)). The second group received 150 μg/kg of the OX26-SA vector alone. The third group received 25 μg/kg of unconjugated bio-bFGF, and the last group was treated with the conjugate of bio-bFGF/OX26-SA, equivalent to 25 μg/kg of bFGF. Each bar represents the mean ±SD of the group. *** p<0.01 as compared to the vehicle group.

FIG. 7 shows the improvement of neurologic deficit scores by bio-bFGF/OX26-SA. The treatment schedule is the same as FIG. 6. Each bar represents the mean ±S.D. of the group. **, p<0.01, ***, p<0.001 compared with the vehicle group.

FIG. 8 depicts the amino acid sequence of a fusion protein of human FGF-2 (SEQ. ID. NO. 5), which is fused to the carboxyl terminus of the heavy chain (HC) of a humanized monoclonal antibody to the human insulin receptor (HIRMAb). The HC is comprised of a variable region (VH) and a constant region (CH); the CH is further comprised of 3 sub-regions, CH1 (SEQ. ID. NO. 1), CH2 (SEQ. ID. NO. 3), and CH3 (SEQ. ID. NO. 4); the CH1 and CH2 regions are connected by a 12 amino acid hinge region (SEQ. ID. NO. 2). The VH is comprised of 3 framework regions (FR) and 3 complementarity determining regions (CDR). The amino acid sequence of the 3 CDRs and the 3 FRs of the VL are blocked, as these are unique to the particular HIRMAb. The amino acid sequence shown for the CH is well known in existing databases and corresponds to the CH sequence of human IgG1.

### DETAILED DESCRIPTION OF THE INVENTION

Compositions in accordance with the present invention include basic fibroblast growth factor that has been conjugated to a BBB transport vehicle, including use of avidin-biotin technology. When avidin-biotin technology is used, the transport vehicle is composed of a blood brain barrier targeting agent that is bound to avidin or streptavidin. The composition is intended for use in treating disorders of the brain, such as cerebral ischemia. It may also be used in vitro or in vivo as a pharmaceutical or diagnostic agent whenever it is desirable to transport biologically active bFGF by receptor mediated transcytosis.

Basic fibroblast growth factor is commonly referred to as bFGF or FGF-2. Although bFGF that is obtained from non-human sources may be used, it is preferred that human bFGF be used. Human bFGF is available from a wide variety of commercial sources. The bFGF used in the following examples was obtained from Scios Inc. (Sunnyvale, Calif.). Human bFGF is also available from other manufacturers, such as Sigma Chemical Co. Human bFGF may also be prepared according to well-known recombinant techniques, if desired, following the routine cloning or synthesis of the bFGF gene.

The bFGF is biotinylated according to known procedures used to biotinylate other drugs and diagnostic agents. It is preferred that the bFGF be monobiotinylated. Specifically, the molar ratio of biotin to bFGF should be about 1 to 1. The bFGF may be polybiotinylated for a particular application, if desired. However, if the bFGF is modified so as to contain 2 or more biotin groups, and this multi-biotinylated bFGF is mixed with the TA-SA or TA-avidin, then high molecular weight aggregates will form, owing to the multivalency of SA or avidin binding of biotin. The increase in size of the final BBB conjugate may not be suitable for in vivo use due to possible immunological attack and rapid clearance of the aggregated conjugate from the blood stream. Aggregation is eliminated by attaching only 1 biotin group to the bFGF.

The transport vehicle is formed by conjugating a BBB targeting agent (TA) to avidin or streptavidin which is a bacterial analog of avidin. The terms “avidin” and “streptavidin”, as used herein, are intended to cover not only avidin and streptavidin, but also to cover chemical or genetically modified avidin or streptavidin compounds that are still capable of providing a strong conjugation bond with biotin. Either avidin or streptavidin could be used in humans, but the protein that gives the least immunologic reaction in humans is the preferred composition. Both avidin and streptavidin are foreign proteins. However, humans are likely immune tolerant to avidin, owing to the high content of avidin in Western diets, and to the immune tolerance induced by oral antigen feeding.

The blood-brain barrier (BBB) targeting agent may be any of the known vectors that undergo receptor mediated transport across the BBB via endogenous peptide receptor transport systems localized in the brain capillary endothelial plasma membrane, which forms the BBB in vivo. Preferred targeting agents include insulin, transferrin, insulin-like growth factor (IGF), leptin, low density lipoprotein (LDL), and the corresponding peptidomimetic monoclonal antibodies that mimic these endogeneous peptides. Peptidomimetic monoclonal antibodies bind to exofacial epitopes on the BBB receptor, removed from the binding site of the endogenous peptide ligand, and “piggyback” across the BBB via the endogenous peptide receptor-mediated transcytosis system. Peptidomimetic monoclonal antibodies are species specific. For example, the OX26 murine monoclonal antibody to the rat transferrin receptor is used for drug delivery to the rat brain (Pardridge et al, 1991). The OX26 antibody to the rat transferrin receptor does not work in other species, including mice (Lee et al, 2000). Accordingly, the OX26 antibody to the rat transferrin receptor would not be used in humans. The OX-26 monoclonal antibody, as described in the following examples, is a suitable transferrin receptor targeting agent for rats. Monoclonal antibodies to the human insulin receptor (HIR) are preferred for delivering bFGF to the human brain. It is preferred that “humanized” monoclonal antibodies be used, and not the original mouse form of the antibody. Exemplary, humanized monoclonal antibodies to the human insulin receptor that are particularly well-suited for use in the present invention are described in detail in copending application UC No. 2003-078-1 (Attorney Docket No. 0180-0038) that is owned by the same assignee as the present application and which has been filed on the same day as this application). The contents of this application are hereby specifically incorporated by reference. Other exemplary targeting agents include the rat 8D3 or rat RI7-217 monoclonal antibody to the mouse transferrin receptor for drug delivery to mouse brain (Lee et al, 2000), or murine, chimeric or humanized antibodies to the human or animal transferrin receptor, the human or animal leptin receptor, the human or animal IGF receptor, the human or animal LDL receptor, the human or animal acetylated LDL receptor.

The targeting agent is conjugated to streptavidin or avidin using generally known techniques, including chemical coupling methods or genetic engineering. In an exemplary procedure for the chemical coupling method, the monoclonal antibody targeting agent is thiolated and then mixed with an activated form of streptavidin or avidin. The resulting conjugate of targeting agent and streptavidin or avidin is then isolated and purified. A preferred chemical for activating streptavidin or avidin is m-maleimidobenzoyl N-hydroxysuccimidyl ester (MBS). Other known activators may also be used. It is preferred that a sufficient amount of streptavidin or avidin be reacted with the targeting agent to provide a molar ratio of streptavidin or avidin to targeting agent that is greater than 1 to 1. Molar ratios on the order of 3 to 1 are preferred. Alternatively, the TA-avidin or TA-SA conjugate may be formed by genetic engineering, since the genes encoding the TA, the avidin, or the SA are all available. In this approach the SA or avidin gene is fused to part of the TA gene corresponding to either the amino or carboxyl terminus of the TA protein. The new fusion gene is used to transfect prokaryotic or eukaryotic expression systems to produce the new TA-avidin or TA-SA fusion protein.

The biotinylated bFGF (bio-bFGF) is conjugated with the targeting agent/streptavidin or avidin complex 1 by combining the two ingredients at room temperature in accordance with generally known techniques for binding two compounds together using avidin-biotin linkages. The relative amounts of bio-bFGF and TA-SA are chosen such that the resulting molar ratio of bFGF to targeting agent is between about 1 to 1 and 1 to 4. A preferred molar ratio of the biotinylated bFGF to targeting agent-avidin or targeting agent-SA is about 3 to 1. In this approach, the bio-bFGF is prepared and stored in one vial. In parallel, the TA-avidin or TA-SA is prepared and stored in a second vial. Both vials may be stored either at temperatures <0 degrees, or may be stored at 4° C. with appropriate bacteriostatic agents. The two vials are mixed just prior to intravenous administration. Owing to the very high affinity of avidin or SA binding of biotin (dissociation constant is femtomolar and the dissociation half-time is 3 months), there is rapid formation of the entire bio-bFGF/TA-SA or bio-bFGF/TA-avidin complex, which is stable in the bloodstream and during transport across the BBB in vivo.

The bio-bFGF/TA-SA conjugate is preferably administered by intravenous injection (i.v.). Any pharmaceutical carrier may be used that is designed for i.v. injection and which does not adversely affect the biological activity of the bFGF. Exemplary carriers include saline or water buffered with acetate, phosphate, TRIS, or a variety of other buffers, with or without low concentrations of mild detergents, such as one from the Tween series of detergents. The dosage of bio-bFGF/TA-SA conjugate will vary depending upon the particular neurological condition being treated. Dosage levels will typically range from 1 μg/kg to 50 μg/kg of bFGF per day. Higher dosage levels should be avoided due to possible adverse reactions due to the toxicity of bFGF. The preferred dosage range is between 5-25 μg/kg.

The bio-bFGF/TA-SA conjugate is especially well suited for use as a neuroprotective agent in treating cerebral ischemia. The conjugate should be administered to the patient as soon as possible. The conjugate should be administered within 3-5 hours after the onset of ischemia. It was found that the bio-bFGF/TA-SA conjugate was more effective as a neuroprotective agent and provided increased reduction in the size of cerebral infarctions when administered within the first 1-2 hours after the start of ischemia. The preferred initial dosage level is about 5-25 μg/kg of bFGF. This amount may be increased or decreased depending upon the severity of the ischemia and the time since the onset of ischemia.

The bFGF may be attached to the BBB TA without the use of avidin-biotin technology by using genetic engineering, whereby the gene encoding for bFGF is fused to the region of the TA gene corresponding to the amino or carboxyl terminus of the TA protein. Such genetic engineering is well known, as the gene for FGF2 has been fused to the region of the gene corresponding to the amino terminus of the plant toxin, saporin (McDonald et al, 1996). In another application, acidic FGF was fused to the carboxyl terminus of the Fe fragment of a human IgG (Dikov et al, 1998). In both applications, the biological activity of the bFGF was retained despite the genetic engineering and fusion to the second protein. For transport of bFGF across the human BBB, the bFGF gene would be fused the region of the TA gene corresponding to the amino or carboxyl terminus of the TA protein to produce a TA-bFGF fusion protein. This TA-bFGF fusion protein would be functionally equivalent to the bio-bFGF/TA-SA or bio-bFGF/TA-avidin complex.

The following examples are provided to provide additional details and teachings with respect to the present invention.

### EXAMPLE 1

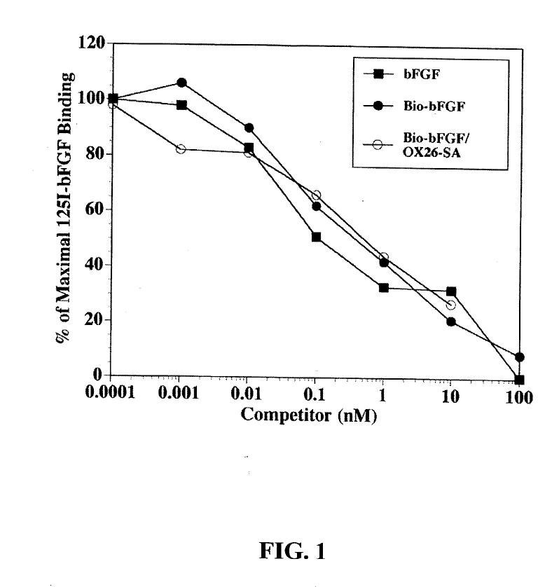

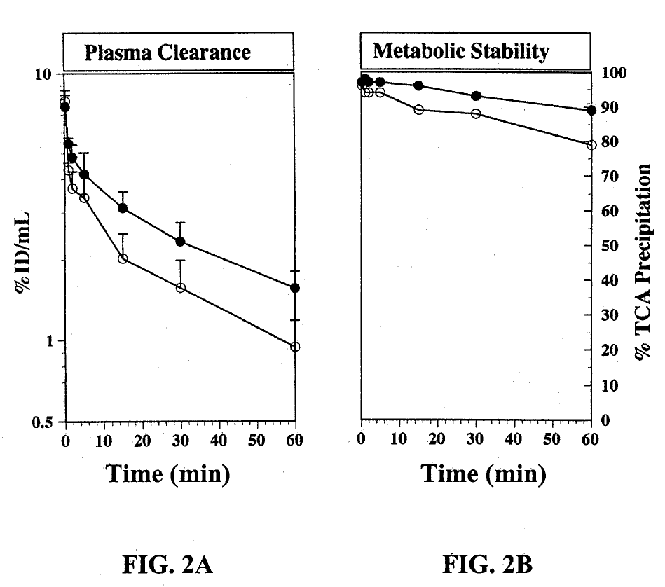

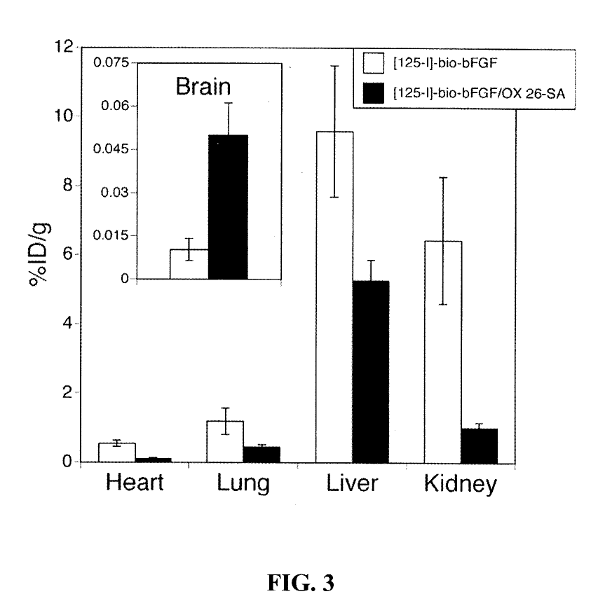

This example shows that the brain uptake of bFGF is increased following intravenous administration if this peptide is re-formulated to enable receptor-mediated transport across the BBB. The avidin-biotin technology is used to conjugate bFGF to the OX26 mouse monoclonal antibody (Mab) to the rat transferrin receptor to trigger receptor-mediated transport across the BBB (Pardridge, 1991). A conjugate of the OX26 Mab and streptavidin (SA) is prepared and is designated OX26/SA. In parallel, bFGF is monobiotinylated to form bio-bFGF, and the complex of bio-bFGF and OX26/SA is designated bio-bFGF/SA-OX26. The bio-bFGF/OX26-SA conjugate is shown to maintain high binding affinity for the bFGF receptor in cultures of BHK-21 cells (FIG. 1). The bio-bFGF/OX26-SA had a decreased peripheral organ distribution (FIGS. 2-3), and an increased brain uptake relative to unconjugated bio-bFGF following intravenous injection (FIG. 2). The enhanced brain uptake of bio-bFGF/OX26-SA was confirmed with an internal carotid artery perfusion method (FIG. 3).

Materials

Male Sprague-Dawley (SD) rats weighing 250-310 grams were purchased from Harlan Sprague Dawley, Inc. (Indianapolis, Ind.). BHK-21 (baby hamster kidney derived cells) were provided by the American Type Culture Collection (Manassas, Va.). Recombinant human basic fibroblast growth factor (bFGF) was provided by the Scios Inc. (Sunnyvale, Calif.). Na¹²⁵I with specific activity of 2050 Ci/mmol. [125I] labeled bFGF (specific activity of 800-1400 Ci/mmol), and [¹⁴C]-sucrose (specific activity of 475 mCi/mmol) were purchased from Amersham (Arlington Heights, Ill.). Biotin-XX-NHS was obtained from CalBiochem (La Jolla, Calif.), where NHS is N-hydroxysuccinimide, and -XX- is bis-aminohexanoyl, 2-Iminothiolane (Traut's reagent), m-maleimidobenoyl-N-hydroxysuccinimide ester (MBS), and BCA protein assay reagents were purchased from Pierce (Rockford, Ill.). Recombinant streptavidin and all other chemicals were obtained from Sigma (St. Louis, Mo.).

Biotinylation of bFGF

Recombinant human bFGF (Scios Product Code P8504, MW 16,400), 43 nmol, was added to 300 μl of 0.05 M NaHCO₃ (pH 8.3), and mixed with 430 nmol of biotin-XX-NHS in 12.3 μl dimethyl sulfoxide. The reaction proceeded at room temperature for I hour with gentle shaking, and was stopped by the addition of 10 μmol of glycine. The products were transferred into a dialysis bag (Spectrum Laboratories, molecular weight cutoff of 6000-8000 Da), and were dialyzed three times against 1 liter of fresh 10 mM phosphate buffer, pH 7.4 at 4° C. for 12 hours. The final yield of bio-bFGF, as determined by Pierce protein assay, was approximately 85% of bFGF used. The molar ratio of biotin incorporated into bFGF protein, based on the (4′-hydroxyazobenzene-2-carboxylic acid) (HABA) assay was 1 to 1.

Iodination of Bio-bFGF

Biotinylated bFGF (bio-bFGF) was iodinated according to the method reported by Neufeld and Gospodarowicz (1985). Briefly, 1.0 nmol of bio-bFGF in 60 μl of 0.2 M phosphate buffer (pH 7.2) was added to Iodogen-coated tubes, followed by the addition of 2 mCi of [¹²⁵I] Na (1 nmol), and the mixture was allowed to react at room temperature for 15 minutes. The reaction was stopped by the addition of 60 μl of 0.1% sodium metabisulfite and 30 μl of 0.1 mM KI. The products were applied to a pre-packed heparin affinity column (Pierce Chemical, Rockford, Ill.) containing 0.7 ml of the slurry, which had been equilibrated with 10 ml of wash buffer (20 mM NaH₂PO₄, 0.6 M NaCl, pH 7.2). The column was washed with 10 ml of the wash buffer, and [¹²⁵I] labeled bio-bFGF was eluted with 1.5 ml of elution buffer (20 mM NaH₂PO₁ 2.0 M NaCl, pH 7.2). Fractions (0.25 ml each) were collected, and radioactivity was counted using a Beckman gamma counter. The specific activity of [¹²⁵I]-bio-bFGF was approximately 170 μCi/mmol with a TCA perceptibility of >98%. The peak fractions were pooled, and gelatin was added to a final concentration of 0.2%. The [¹²⁵I]-bio-bFGF was stored at −20° C.

Synthesis of OX26-SA Conjugate

The OX26/SA conjugate was prepared as described previously (Kang and Pardridge, 1994). Briefly, 20 mg of murine OX26 monoclonal antibody was thiolated with a 10:1 molar ratio of 2iminothiolane. In parallel, 7 mg of recombinant streptavidin (SA) was activated with a 20:1 molar ratio of m-maleimidobenzoyl N-hydroxysuccimidyl ester (MBS). At the end of the OX26 thiolation and SA activation, the two samples were pooled and allowed to stand at room temperature for 3 hours for conjugation. The conjugate was labeled with 2.5 μCi of [³H]-biotin and was purified on a 2.6×92 cm column of Sephacryl S300HR (Pharmacia) followed by elution in 0.01 M Na₂HPO₄/0.15 MnaCl/pH 7.4 0.05%. Tween-20 at 30 ml/h, and 3 ml fractions were collected. The conjugate peak eluted between fractions 70-89 and was well separated from unconjugated SA (fractions 98-107). The number of biotin binding sites per OX26/SA conjugate was approximately three, as determined using a [³H]-biotin binding assay (Kang and Pardridge, 1994).

BFGF Radioceptor Binding Assay

The radioreceptor binding assay was performed as reported by Neufeld and Gospodarowicz (1985). The BHK-21 cells (10⁵/well) were sub-cultured for one day using poly-D-lysine coated 24-well cluster dishes, and maintained with DMEM and 10% fetal bovine serum and antibiotics. The cells were washed twice with 1.0 ml/well of cold DMEM containing 0.2% gelatin. The cells were incubated in triplicate at 4° C. for 4 hours with [¹²⁵I]-bFGF (6000 dpm/well) and graded doses of either native bFGF (final concentrations from 1 pM to 200 nM), or corresponding doses of bio-bFGF or the conjugate, bio-bFGF/OX26-SA, in a total volume of 0.5 ml/well. At the end of the incubation, the medium was aspirated, and the cells were washed three times with 0.5 ml/well of cold DMEM containing 0.1% BSA, followed by the addition of 0.5 ml/well of 1% Triton-X-100 for cell lysis. The radioactivity was counted with a Packard liquid scintillation counter (Packard Instrument, Downer's Grove, Ill.). The data was expressed as a % of maximal binding, plotted vs. the concentration of bFGF using Deltagraph 4.5 software, and the bFGF concentration that caused 50% inhibition of binding (IC₅₀) was graphically determined.

Pharmacokinetics

Rats were anesthetized with 100 mg/kg ketamine and 2 mg/kg xylazine intraperitoneally. The left femoral vein was cannulated with a PE₅₀ cannula and injected with a 0.2 ml Ringer-Hepes solution (pH=7.4) containing 4 μCi (0.023 nmol) of [¹²⁵I]-bio-bFGF conjugated to either 0 or 6.6 μg (0.033 nmol) of OX26/SA. Conjugation was accomplished by simply mixing the bio-bFGF and OX26-SA vials together prior to injection. Owing to the very high affinity of SA binding of biotin, there was rapid formation of the bio-bFGF/OX26-SA complex.

Blood samples (0.25 ml) were collected via heparinized PE₅₀ cannula implanted in the left femoral artery at 0.25, 1, 2, 5, 15, 30, and 60 minutes after IV injection. Blood volume was replaced with an equal volume of saline. After the end of 60 minutes, the animals were decapitated for the removal of the brain and four peripheral organs (liver, kidney, heart, and lung). The plasma and organ samples were solubilized with Soluene-350 (Packard Instrument Company, Downer's Grove, Ill.) and neutralized with glacial acetic acid prior to liquid scintillation counting. The metabolic stability of the [¹²⁵I]-bio-bFGF or [¹²⁵I]-bio-FGF/OX26-SA was determined by TCA precipitation of 50 μl aliquots of plasma removed at each time point (FIG. 2).

Pharmacokinetic parameters were calculated by fitting the plasma TCA precipitable radioactivity data to a biexponential equation:

A(t)=Aₜe^{-k}^{₁}^{t}+A₂e^{-k}^{₂}^{t}

where A(t)=% injected dose (ID)/ml plasma. The biexponential equation was fit to plasma data using a derivative-free non-linear regression analysis (PAR-BMDP, Biomedical Computer P-Series, developed at the UCLA Health Sciences Computing Facilities). The data were weighted using weight=1/(concentration)², where concentration=% ID/ml plasma. The organ volume of distribution (V_{d}) of the [¹²⁵I]-bio-bFGF or its conjugate with OX26-SA at 60 minutes after IV injection was determined from the ratio of disintegration/minutes (dpm)/g tissue divided by the dpm/μl of the terminal plasma. The pharmacokinetic parameters such as plasma clearance (CL), initial plasma volume (V_{C}), steady state volume of distribution (V_{SS}) area under the plasma concentration curve (AUC), and mean residence time (MRT) were determined from the A₁, A₂, K₁, and K₂, as described previously (Kang and Pardridge, 1994). The organ clearance or permeability surface area (PS) product was determined as previously described by Pardridge et al., 1994. The organ uptake, expressed as percentage dose (ID) per gram organ, was calculated from:

% ID/g=PS[AUC]

The pharmacokinetic parameters are given in Table 1, and show that the systemic clearance of the bFGF is decreased 40% following conjugation to OX26-SA; this decrease in systemic clearance reflects the decrease in uptake of the bFGF by peripheral organs, as shown in FIG. 3. Despite this decrease in uptake of bFGF by peripheral organs, the bFGF uptake by brain is actually increased (FIG. 3), as reflected by the >300% increase in BBB PS product for bFGF (Table 1).

Internal Carotid Artery Perfusion Technique

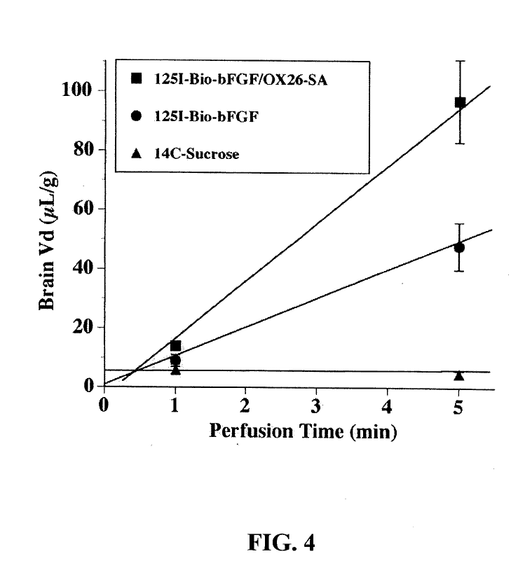

Rats were anesthetized with ketamine/xylazine and the right internal carotid artery was cannulated with a PE₁₀/PE₅₀ tubing after electrocoagulation of the ipsilateral superior thyroid, occipital and pterygopalatine arteries, as described previously (Wu et al., 1996). Prior to the perfusion, the ipsilateral common carotid artery was ligated, and the internal carotid artery was perfused with Krebs-Henseleit buffer containing 0.1% rat serum albumin (RSA), 0.5 μCi/ml of [¹²⁵I]-bio-bFGF (2.92 nM) with or without conjugation to OX26/SA (4.15 nM), and 2.0 μCi/ml of [¹⁴C]-sucrose at a perfusion rate of 1.2 ml/min. The [¹²⁵I]-bio-bFGF was iodinated on the same day with a TCA precipitability of >98%. The pH of the perfusate was adjusted to 7.4 after gassing with 95% O₂-5% CO₂, passed through a 0.45 μm Millex-HV filter (Millipore, Bedford, Mass.), and maintained in a 37° C. water bath. The blood volume was maintained relatively constant by simultaneously withdrawing femoral arterial blood at a rate of 1.0 ml/min. At the end of either one or more five minutes of perfusion, the animals were decapitated. The ipsilateral brain hemisphere was removed, and cut into three pieces. The first piece was used for direct [¹²⁵I] radioactivity by a Beckman gamma counter. The second piece was solubilized in Soluene-350 for liquid scintillation counting of [¹⁴C] activity using an energy window between 30 and 156 keV. The last piece of the brain was homogenized for separation of postvascular supernatant and capillary pellet by the capillary depletion technique (Triguero et al., 1990). This work shows the conjugation of the bFGF to the OX26-SA vector increases BBB transport of the bFGF at levels at least 100% above that of the unconjugated bFGF (FIG. 4).

Binding Affinities of bFGF and Its Analogs

As shown in FIG. 1, the native bFGF, the bio-bFGF, and the bio-bFGF/OX26-SA conjugate displaced the [¹²⁵I]-bFGF binding to the BHK-21 cells in a concentration-dependent manner. The IC₅₀ values were estimated to be 0.12, 0.40, and 0.56 nM for bFGF, bio-bFGF, and bio-bFGF/OX26-SA, respectively. The OX26-SA (200 nM) in the absence of bio-bFGF had no effect on the [¹²⁵I]-bFGF binding. The non-specific binding of [¹²⁵I]-bFGF to the culture plates in the absence of BHK-21 cells were approximately 11% of the total binding. These receptor assays yield the unexpected finding that bFGF still binds with high affinity to its cognate receptor despite biotinylation and conjugation of the bio-bFGF to the OX26-SA vector. In contrast, other neurotrophins loose all affinity for the targeted receptor following mono-biotinylation and conjugation to OX26-SA (Deguchi et al, 1999), because attachment of the peptide to the OX26-SA causes steric hindrance of the receptor/peptide binding reaction. Conversely, the retention of the high affinity receptor binding of the bFGF to its cognate receptor, despite conjugation to the transport vector, illustrates the novel features of the bFGF-TA complex.

Pharmacokineties of [¹²⁵I]-bio-bFGF with or without Conjugation to OX26-SA

The time course of clearance from blood of the [¹²⁵I]-bio-bFGF or [¹²⁵I]-bio-bFGF/OX26-SA conjugate is shown in FIG. 2A. The TCA precipitation of [¹²⁵I]-bio-bFGF and the [¹²⁵I]-bio-bFGF/OX26-SA conjugate was 79±1% and 89±2%, respectively, at the end of 60 minutes (FIG. 2B).

The pharmacokinetic parameters for [¹²⁵I]-bio-bFGF or the [¹²⁵I]-bio-bFGF/OX26-SA conjugate were determined from the plasma profile data in FIG. 2A, and are listed in Table 1. The plasma AUC of the conjugate at 60 minutes was increased 50% (161±19 vs. 111±26% ID min/ml) as compared to the AUC of unconjugated [¹²⁵I]-bio-bFGF. FIG. 3 shows predominant distribution of [¹²⁵I]-bio-bFGF after IV injection in the liver and kidney, and less uptake in other peripheral organs, such as the heart and lung. The rapid uptake of the [¹²⁵I]-bio-bFGF by peripheral tissues-was decreased by conjugation to OX26-SA conjugate (FIG. 3), and this resulted in an increase in the plasma AUC (Table 1). The brain V_{d} of the bio-bFGF was not significantly different from the brain plasma volume. As a result, the BBB permeability surface area (PS) product of the bio-bFGF could not be computed (Table 1). The brain uptake (% ID/g) of [¹²⁵I]-bio-bFGF/OX26-SA was 5-fold higher than that of [¹²⁵I]-bio-bFGF (FIG. 3).

BBB Transport after Internal Carotid Artery Perfusion (ICAP)

As shown in FIG. 4, the brain V_{d} of [¹⁴C]-sucrose, a plasma volume marker, was unchanged at either one or five minutes of ICAP, and always under 5 μl/g, indicative of an intact BBB during the period of the ICAP study. After 5 minutes of ICAP, the brain V_{d} of [¹²⁵I]-bio-bFGF was 10-fold above the plasma volume, indicative of BBB transport of bio-bFGF. Conjugation of [¹²⁵I]-bio-bFGF to the OX26-SA drug delivery vector produced a nearly 2-fold increase in the brain V_{d} after 5 minutes of ICAP, indicating that the OX26-SA drug delivery vector enhanced the BBB transport of bio-BFGF. The 5 minute brain homogenate obtained after perfusion with the [¹²⁵I]-bio-bFGF conjugated to the OX26-SA was analyzed with the capillary depletion method (Triguero et al., 1990). The brain capillary depletion study showed approximately 67% of the [¹²⁵I] radioactivity was in the postvascular supernatant, while the remaining 33% of the [¹²⁵I] radioactivity was in the capillary pellet. This shows transcytosis of bio-bFGF/OX26-SA across the BBB.

TABLE 1
Pharmacokinetic parameters and brain uptake of [¹²⁵I]-bio-bFGF or
[¹²⁵I]-bio-bFGF/OX26-SA at 60 minutes after intravenous injection in rats

| Parameter | Bio-bFGF | Bio-bFGF/OX26-SA |
|---|---|---|
| A₁ (% ID/ml) | 7.99 ± 1.47 | 4.07 ± 0.21 |
| A₂ (% ID/ml) | 3.14 ± 0.87 | 4.16 ± 0.46 |
| K₁ (min⁻¹) | 2.29 ± 1.13 | 1.00 ± 0.19 |
| K₂ (min⁻¹) | 0.0212 ± 0.0025 | 0.0172 ± 0.0013 |
| t¹_{1/2} (min) | 0.53 ± 0.27 | 0.75 ± 0.14 |
| t²_{1/2} (min) | 34 ± 4 | 41 ± 3 |
| AUC₀₋₆₀ (% ID min/ml) | 111 ± 26 | 161 ± 19 |
| AUC₀₋₀₀ (% ID min/ml) | 155 ± 37 | 249 ± 34 |
| V_{C} (ml/kg) | 35 ± 8 | 46 ± 5 |
| V_{SS} (ml/kg) | 119 ± 28 | 89 ± 13 |
| CL_{SS} (ml/min/kg) | 2.63 ± 0.73 | 1.57 ± 0.31 |
| MRT (min) | 46 ± 6 | 58 ± 4 |
| Brain PS (μl/g/min) | <0.08 | 0.264 ± 0.019 |

Parameters computed from the serum radioactivity profile in FIG. 2.

The results of the present example support the following conclusions. First, bFGF can be monbiotinylated, and conjugated to the OX26-SA vector, and still retains receptor-binding affinity in the nM range (FIG. 1). Second, conjugation of [¹²⁵I]-bio-bFGF to the OX26-SA vector results in brain drug targeting as the uptake of bFGF by peripheral tissues is decreased, while there is a >5-fold increase in brain uptake of the conjugate compared to free [¹²⁵I]-bio-bFGF (FIG. 3, Table 1).

In the radioreceptor binding study, baby hamster kidney-derived cell (BIIK-21) cultures were used as a model because of the presence of high density of bFGF binding sites on the surface of these cells (Gospodarowicz, 1984; Neufeld and Gospodarowiicz, 1985, 1986). One of the confounding factors in the binding assay is internalization of bFGF through both receptor-mediated and heparan sulfate-mediated mechanisms (Roghani and Moscatelli, 1992). To minimize the internalization, all the incubations were carried out at 4° C. for 4 hours. The non-specific binding in this study was approximately 11%, which is comparable to the results using the same model reported by Neufeld and Gospodarowicz (1985, 1986). The IC₅₀ value of native bFGF obtained in this study (0.12 nM) was consistent with the previously reported K_{D} of 0.27 nM (Neufeld and Gospodarowicz, 1985). As shown in FIG. 1, both bio-bFGF and bio-bFGF/OX26-SA inhibited the binding of [¹²⁵I]-bFGF in a concentration-dependent manner with IC₅₀ values in a low nanomolar range, indicating that there is a retention of binding affinity after monobiotinylation and conjugation of bFGF to OX26/SA. The biotin is attached to the ω-amino group of a surface lysine residue, and the radioreceptor studies indicate this modification does not interfere with bFGF binding to its receptor.

The unconjugated [¹²⁵I]-bio-bFGF is rapidly taken up by peripheral organs such as liver and kidney with a negligible brain uptake after IV injection (FIG. 3). Conjugation of [¹²⁵I]-bio-bFGF to the OX26/SA vector resulted in a decrease in organ uptake by all peripheral tissues, in parallel with at least 5-fold increase in brain uptake (FIG. 3 and Table 1). The actual increase in brain uptake of bFGF caused by conjugation to the Mab is >5-fold, because the brain uptake of the unconjugated bFGF shown in FIG. 3 reflects, in part, the brain uptake of [¹²⁵I]-labeled metabolites generated by the peripheral degradation of unconjugated bFGF. The enhanced brain uptake following conjugation of bFGF to OX26/SA was confirmed in the ICAP study (FIG. 4). Conjugation of bFGF to the OX26/SA causes a 2-fold increase in brain uptake following internal carotid artery perfusion (FIG. 4), but a >5-fold increase in brain uptake following intravenous (IV) injection (FIG. 3). Conjunction of bFGF to the BBB drug delivery vector has two beneficial effects: (a) increase in BBB PS product (Table 1) and (b) decreased uptake by peripheral issues (FIG. 3), which causes an increase in the plasma AUC (Table 1). Both the increased PS product and increased plasma AUC have additive effects to increase the brain uptake (% ID/g) of the bFGF following IV administration. However, the AUC factor is held constant in the ICAP experiment, which explains why the brain uptake is increased to a greater extent following IV, as opposed to ICAP administration.

The targeting of the bFGF conjugate to the brain, and away from peripheral tissues is desired, because bFGF has variety of physiological activities in the periphery, including vasodilation, mitogenic effects, and angiogenesis (Bikfalvi et al. 1997). Repeated intravenous administration of bFGF (100 μg/kg/day for 4 weeks) in both rats and monkeys resulted in anemia, hyperostosis, and reversible glomerular injury (Mazue et al., 1992; 1993). In two Phase-I clinical trials, bFGF was shown to produce dose-dependent leukocytosis in patients with acute ischemic strokes (Clark et al., 2000; Lahman et al., 2000). Therefore, the brain drug targeting strategy in accordance with the present invention is needed to reduce the peripheral side effects of bFGF, while selectively promoting pharmaceutical effects within the CNS. Data presented in this example show that conjugation of bFGF to a BBB transport vector increased the metabolic stability in the plasma (FIG. 2), decreased peripheral organ uptake (FIG. 3), and increased the brain uptake after an IV injection (FIG. 3). These combined effects enable neuroprotection in the brain at reduced systemic doses, owing to the increased therapeutic index of the conjugate compared to unconjugated bFGF.

The carotid artery perfusion study demonstrated a modest transport of [¹²⁵I]-bio-bFGF across the BBB (FIG. 4) in the absence of a BBB drug delivery vector. This finding is consistent with the results reported by Deguchi et al. (2000), and may explain why intravenous infusion of high doses of bFGF (150 μg/kg) in the rat model of focal cerebral ischemia reduces brain infarct volume (Fisher et al., 1995; Jiang et al., 1996; Tatlisumak et al., 1996; Ren and Finklestein, 1997). However, the BBB transport of [¹²⁵I]-bio-bFGF without a BBB delivery system is modest, and difficult to measure following intravenous administration as shown in Table 1. Similarly, Fisher et al. (1995) reported that the brain uptake of [¹²⁵I]-bFGF in the rat after peripheral administration was not measurable by autoradiography unless the BBB was disrupted in a regional brain ischemia model.

In summary, this example shows that the affinity of bFGF for its receptor is retained following biotinylation and conjugation to a BBB drug delivery vector. This conjugation has the dual effect of decreasing the uptake of bFGF by peripheral tissues, and increasing the uptake by the brain. Conjugation of bFGF to the OX26 antibody to the transferrin receptor triggers receptor-mediated transport of bFGF on the BBB transferrin receptor. Both the increased brain uptake and decreased clearance by peripheral tissues augment the therapeutic index of bFGF and enables neuroprotection in the brain following the intravenous administration of lower systemic doses of the peptide. Additional details regarding this example are set forth in Wu et al., 2002.

### EXAMPLE 2

This example demonstrates the neuroprotective effects of bFGF after reformulation and conjugation to a BBB delivery vector in accordance with the present invention. The example uses mixed rat cortical cell culture model in vitro and the permanent middle cerebral artery occlusion model in vivo. The example shows neuroprotection in regional brain ischemia following the delayed intravenous administration of low doses (25 μg/kg) of bFGF, provided that the bFGF is biotinylated and conjugated to a BBB drug-targeting agent in accordance with the present invention.

Materials. Male Sprague-Dawley rats weighing 280 to 320 grams and pregnant rats of 16 day gestation age were purchased from Harlan (Indianapolis, Ind.). Recombinant human bFGF was provided by Scios Inc. (Sunnyvale, Calif.), DMEM (with high glucose), fetal bovine serum (FBS), and antibiotics were purchased from Invitrogen (Carlsbad, Calif.). Biotin-XX-NHS was obtained from Calbiochem (San Diego, Calif.), where NHS is N-hydroxysuccinimide, and XX is bis-aminohexanoyl, 2-Iminothiolane (Tarut's reagent), m-maleimidobenzoyl-N-hydroxysuccinimide ester, and BCA protein assay reagents were purchased from Pierce (Rockford, Ill.). Recombinant streptavidin, 3(4,5-dimethylthiazol-2-yl)-2,5-diphenyltetrazolium bromide (MTT), 2,3,5-triphenyltetrazolium chloride, and all other chemicals were obtained from Sigma-Aldrich (St. Louis, Mo.).

Mixed Rat Cortical Cell Cultures. Mixed rat cortical cells were cultured according to Cazevieille et al. (1993). Briefly, fetal brain was obtained from two pregnant rats of 16-day gestation age. Bilteral forebrain cortices were removed into 2 ml of ice-cold Hepes-buffered saline solution containing 0.05% trypsin. The tissue masses were dissected using microscissors. At the end of incubation in a water bath with gentle shaking at 37° C. for 30 minutes, trypsin inhibitor was added to a final concentration of 0.1%. After standing at room temperature for 20 minutes, the supernatant was aspirated, and the pellets were suspended in DMEM supplemented with 10% FBS and antibiotics. After standing at room for 15 minutes the cell suspension was transferred to a sterile tube, and the tissue pellets were discarded. The cells were plated into 24-well cluster dishses (Costar Corp., Cambridge, Mass.), which were precoated with 0.1 mg/ml poly-L-lysine, at a density of 10⁶ cells/well in 1.0 ml of DMEM supplemented with 10% FBS and antiobiotics. The cultures were maintained at 37° C. with 5% CO₂/95% air and saturating humidity. The medium was changed twice a week.

Bio-bFGF/OX26-SA Conjugate. The bio-bFGF/OX26-SA conjugate was made in the same manner as Example 1. In addition, the bFGF, bio-bFGF and OX26-SA that were used in this Example were the same as in Example 1.

Hypoxic Insult and MTT Assay. In vitro neuroprotective effect of bFGF analogs was assessed using the MTT assay as reported by Dore et al. (1997). The mixed rat frontal cortical cells were cultured for 10 days. One day prior to the test, the medium was replaced with 0.5 ml of serum-free DMEM per well supplemented with 0.1% bovine serum albumin, glucose, and antibiotics, which stops cell division and arrests the cells in the G₀/G₁ phase of cell growth (Kiyokawa et al., 1997). Three graded doses (0.1, 1.0, or 10 ng/ml) either of native bFGF, bio-bFGF, or bio-bFGF/OX26-SA were added to the cultures and incubated for 24 hours. The doses of bio-bFGF/OX26-SA contained 110 ng/ml or 0.55 nmol of OX26-SA. Designated wells were enriched with medium only or corresponding doses of OX26-SA as controls. On the experimental day, the medium was replaced with 0.3 ml of fresh medium per well, and bFGF and its analogs were added at the same concentrations as above. All the cell plates were placed in a custom-made hypoxia chamber maintained in a 37° C. water bath and aerated with 95% N₂/5% CO₂ at a rate of 1.2 l/min for 24 hours. After 4 hours of reoxygenation, 0.5 ml of freshly made MTT solution (0.5 mg/ml, passed through a 0.2 μm filter) was added to each well and followed by 2 hours of incubation in the cell culture incubator. At the end of the incubation, the cells and MTT formazan crystals were solubilized by addition of 1.0 ml of anhydrous isopropanol/0.1 N HCl per well. The total reduced MTT was quantified spectrophotometrically at 570 nm. Background correction was performed with extracts of cells not treated with MTT. The average reduced MTT in designated cell wells without exposure to the hypoxia/reoxygenation insult was considered 100%. To supplement the MTT assay, medium lactate dehydrogenase activity was measured spectrophotometrically. However, enzyme release to the medium was only detected with the combined exposure of the cells to hypoxia and glucose deprivation. This assay was not used further, since glucose was included in the medium to reflect physiologic conditions.

Focal Cerebral Ischemia Model. After fasting overnight, male Sprague-Dawley rats weighing 280 to 320 grams were lightly anesthetized with inhalation of halothane and orotracheally intubated by transillumination as previously reported by Cambron et al. (1995). The animals were artificially ventilated with a mixture of 70% N₂O/30% O₂, and 0.5% halothane at a rate of 90 stroke/min and a volume of 5 ml/stroke. Body temperature was maintained with a Harvard thermal blanket with a rectal probe (Harvard Apparatus, Holliston, Mass.). Systolic blood pressure was measured by a model 29 rat tail arterial pulse amplifier (HTC Inc./Life Science Instruments, Woodland Hills, Calif.). The left femoral artery was cannulated with PE50 tubing from which blood was collected for the measurement of blood pH, pCO₂, and PO₂ using a model 238 pH/blood gas analyzer (Chiron Corp., Emeryville, Calif.). After all the physiologic parameters were stabilized, a ventral midline neck incision was made, and a permanent MCAO was introduced by an intraluminal suture (3-0) (Fisher et al. 1995). The suture was prepared with a rounded tip by heating near a flame, and the size of the tip was checked with a hemocytometer under a microscope to be approximately 0.3 to 0.4 mm. All the physiological parameters were rechecked 10 min after MCAO, and the incision was sutured. The animal was allowed to recover under a heating lamp for 4 hours, and then individually housed in the vivarium with free access to food and water. The animals were anesthetized 24 hours after MCAO with inhalation of halothane and decapitated for removal of the brain. Coronal sections were cut to 2-mm thickness using a rat brain matrix. The brain sections were incubated in 2% 2,3,5-triphenyltetrazolium chloride solution at 37° C. for 30 minutes. The stained sections were fixed in 10% formalin/10 mM phosphate buffer, pH 7.4, and stored at 4° C. The experimental protocol was approved by the UCLA Animal Research Committee.

Treatment Schedule. The rats with MCAO were randomly assigned to four groups, and all rats received pharmacologic treatment via a single femoral vein injection. The first group received 1.2 ml/kg vehicle (10 mM phosphate-buffered saline containing 1% bovine albumin). The second group received 25 μg/kg bio-bFGF and 150 μg/kg OX26-SA. The intravenous injection was administered at 0, 1, 2, and 3 hours after MCAO. One group of animals was treated immediately after MCAO with a lower dose of the conjugate, e.g., 5 μg/kg bio-bFGF coupled to 30 μg/kg OX26SA by a single i.v. injection in 1.2 ml/kg vehicle.

Neurologic Deficit Scores. The neurologic deficit status of the animals was evaluated 2 and 24 hours post-MCAO according to Liu et al. (1999) by a 0- to 5-point scale: grade 0, no visible neurologic deficit; grade 1, failure to extend the right forepaw fully; grade 2, intermittent circling; grade 3, sustained circling without moving forward; grade 4, failure to walk spontaneously with a depressed level of consciousness; and grade 5, death.

Calculation of Infarct Volume. The stained and fixed brain sections were photographed on both sides, using an Epson model 650 digital camera (Epson America, Torrance, Calif.). Infarct areas were measured using the NIH Image Software (version 1.61) and calibrated using a glass circle (10mm diameter) and a square (12×12 mm). The infarct area was corrected to compensate for the effect of brain edema based on the area ratio of the ipsilateral (ischemic) to contralateral (nonischemic) hemispheres. The infarct volume was calculated by summed infarct areas with each section and multiplied by section thickness (2 mm).

Statistical Analysis. Data were presented as the mean ±S.D. of each group of animals. The statistical differences between infarct volumes were assessed with Student's t test for the in vitro data and analysis of variance (ANOVA) using the Bonferroni correction for the in vivo results, as described previously (Zhang and Pardridge, 2001b), p<0.05 was considered statistically significant.

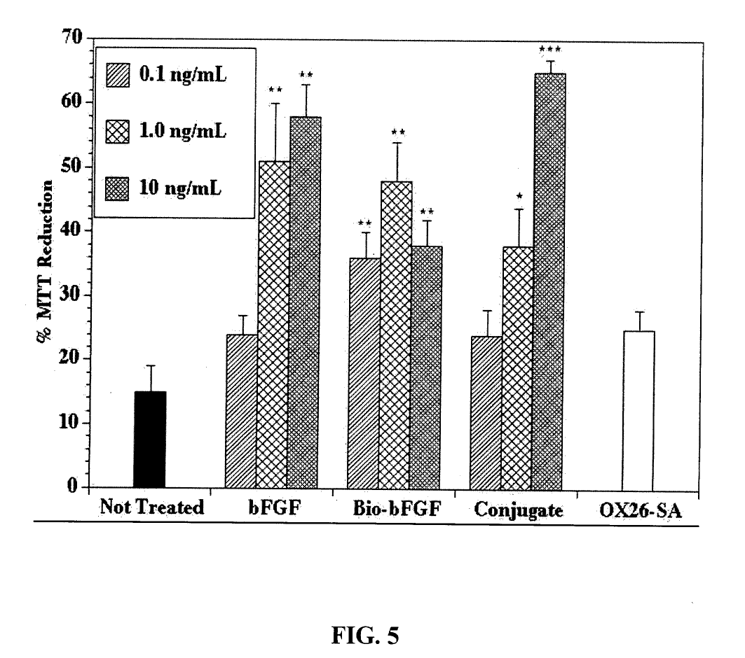

In Vitro Neuroprotection. The hypoxia/reoxygenation insult produced severe inhibition of MTT reduction in the mixed rat cortical cell cultures without treatment (FIG. 5). Pretreatment of the cultures with bFGF resulted in elevated MTT reduction in a dose-dependent manner, and statistically significant effects were observed at the doses of 1.0 ng/ml or greater. The conjugate of bio-bFGF/OX26-SA also showed dose-dependent neuroprotective effects, whereas the vector OX26-SA alone did not have a significant effect compared with the nontreated cultures (FIG. 5).

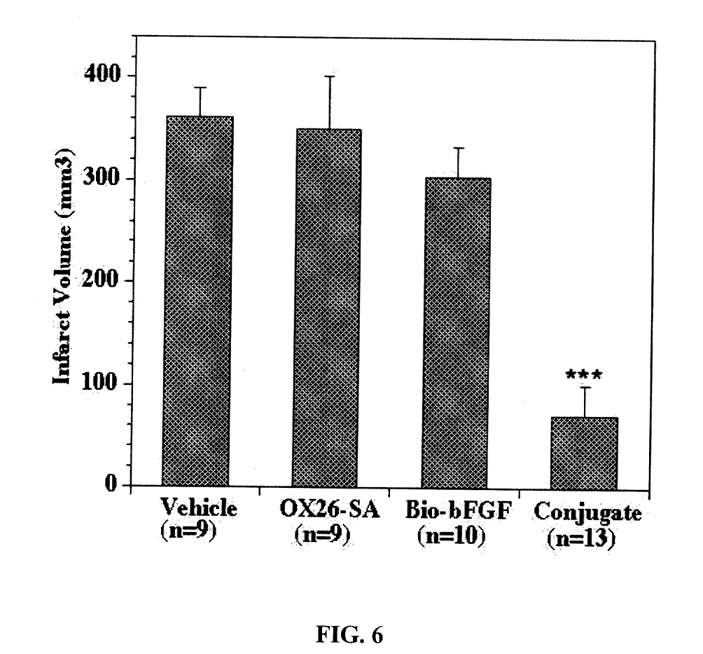

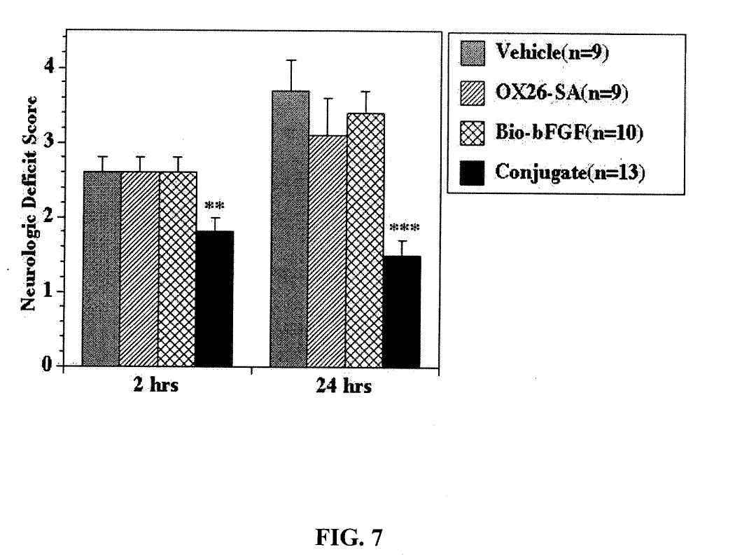

In Vivo Neuroprotection. All physiologic parameters were stable before and 10 minutes after MCAO (Table 2). The infarct volumes in the animals treated immediately after MCAO are shown in FIG. 6. The OX26-SA vector alone (150 μg/kg) did not have any significant effects on the brain infarct volume compared with the vehicle group. Bio-bFGF (25 μg/kg) alone showed a marginal reduction (16%) of infarct volume, but this was not statistically significant compared with the vehicle-treated group. By contrast, a single i.v. injection of the conjugate of bio-bFGF/OX26SA, at a dose equivalent to 25 μg/kg bFGF, resulted in a marked reduction of infarct volume of 80%. FIG. 7 shows shows the neurologic deficit at 2 and 24 hours post-MCAO. Treatment with vehicle, the OX26-SA vector alone, or the bio-bFGF alone caused no changes in neurologic scores (FIG. 7). However, the conjugate of bio-bFGF/OX26-SA significantly improved the neurologic deficit at 2 and 24 hours.

One group of the experimental rats was treated with a lower dose of the conjugate, 5 μg/kg, which is one-fifth the regular dose used in the study, and the infarct volume was reduced by 34% (Table 3). To assess the time window of the neuroprotective effect, the regular dose (25 μg/kg) of bio-bFGF/OX26-SA was given at 1, 2, and 3 hours after MCAO. As shown in Table 3, the treatment with the 1-hour delay produced a significant 66% reduction of infarct volume, and there was significant improvement in the neurologic deficit score at both 2 and 24 hours as well. However, the delay in treatment for either 2 or 3 hours after MCAO showed neither reduction of infarct volume nor improvement of neurologic deficit (Table 3).

This example supports the following conclusions. First, unconjugated bio-bFGF and the bio-bFGF/OX26-SA conjugate retain neuroprotective effects comparable with the native bFGF in the hypoxia/reoxygenation insult assay in the mixed rat cortical cell cultures (FIG. 5). Second, after a single i.v. injection of bio-bFGF/OX26-SA, equivalent to 25 μg/kg bFGF, there is an 80% reduction in stroke volume with significant improvement of neurologic deficit. In contrast, this dose of unconjugated bio-bFGF does not have a statistically significant effect on either stroke volume or neurologic deficit (FIGS. 6 and 7). Third, the neuroprotection of bio-bFGF/OX26-SA is time-dependent with an effective time window of at least 1 hour post-MCAO. Fourth, the potency of the bio-bFGF/OX26-SA conjugate is 500% greater than any other known neurotrophin-TA conjugate. The neuroprotection in the MCAO model achieved with the 5 μg/kg dose of the bio-bFGF/OX26-SA is comparable to the neuroprotection in this model achieved with a 25 μg/kg dose of bio-BDNF/OX26-SA, where BDNF=brain derived neurotrophic factor (Zhang and Pardridge, 2001a). This high potency of the bFGF-TA conjugate, relative to other neurotrophin conjugates, was unexpected and is illustrative of the novel features of the bFGF-TA conjugate.

MTT reduction is an indicator of the mitochondrial activity in living cells and has been used as an indicator of neuronal injury and death (Dore et al., 1997). As shown in FIG. 5, hypoxial/reoxygenation insult produces markedly decreased MTT reduction in the mixed rat forebrain cortical cell cultures. Preincubation with either the native bFGF, free bio-bFGF, or bio-bFGF/OX26-SA conjugate protects the cortical cells against hypoxial/reoxygenation injury in a dose-dependent manner. The effective dose in this in vitro model is 1.0 ng/ml (FIG. 5). The neuroprotective effects of the bio-bFGF/OX26-SA conjugate in tissue culture are consistent with Example 1 showing that the bFGF still binds to the high affinity bFGF receptor despite conjugation to the OX26 antibody. These combined results indicate that the biological activity of bFGF is retained following monobiotinylation and conjugation to OX26-SA.

TABLE 2
Physiological variables

| | | | | Bio-bFGF/ |
|---|---|---|---|---|
| | Vehicle | OX26-SA | Bio-bFGF | OX26-SA |
| | (n-9) | (n-9) | (n-30) | (n-13) |
| Before MCAO | | | | |
| Rectal | 36.4 ± 0.05 | 36.4 + 0.05 | 36.0 ± 0.04 | 36.3 ± 0.04 |
| temperature | | | | |
| pH | 7.39 ± 0.01 | 7.40 ± 0.01 | 7.41 ± 0.01 | 7.41 ± 0.01 |
| pO₂ (mm Hg) | 144 ± 7 | 156 ± 7 | 147 ± 6 | 145 ± 5 |
| pCO₂ (mm Hg) | 44 ± 2 | 44 ± 2 | 42 ± 2 | 43 ± 1 |
| Systolic BP | 115 ± 4 | 99 ± 5 | 97 ± 5 | 102 ± 5 |
| (mm HG) | | | | |
| After MCAO | | | | |
| Rectal | 36.4 ± 0.05 | 36.4 ± 0.04 | 36.3 ± 0.04 | 36.3 ± 0.04 |
| temperature | | | | |
| pH | 7.39 ± 0.01 | 7.40 ± 0.01 | 7.40 ± 0.01 | 7.42 ± 0.01 |
| pO₂ (mm Hg) | 139 ± 4 | 146 ± 5 | 140 ± 5 | 149 ± 7 |
| pCO₂ (mm Hg) | 43 ± 2 | 42 ± 1 | 42 ± 1 | 42 ± 1 |
| Systolic BP | 117 ± 4 | 101 ± 5 | 100 ± 5 | 101 ± 5 |
| (mm Hg) | | | | |

BP = blood pressure

The bFGF/OX26 conjugate is also neuroprotective in vivo in the MCAO model of regional brain ischemia following the delayed intravenous injection of the conjugate (Table 3, FIGS. 6 and 7). In contrast, the unconjugated bFGF is not neuroprotective in the MCAO model following the intravenous injection of a dose of the neurotrophin of 25 μg/kg (FIGS. 6 and 7). Unconjugated bFGF is neuroprotective in the MCAO model providing high doses (135 μg/kg) are administered in a setting where the BBB is disrupted in the region of the infarction (Fisher et al., 1995; Ay et al., 1999). However, in the absence of hyperglycemia-induced vasculopathy (Kawai et al., 1997), the BBB is intact for 4 to 6 hours following regional brain ischemia (Menzies et al., 1993; Belayev et al., 1996; Albayrak et al., 1997). Therefore, if bFGF is to be used as an effective neuroprotective agent in stroke following a delayed intravenous administration, then the neurotrophin must be enabled to cross the BBB in pharmacologically significant amounts. BBB transport is possible if the neurotrophin is conjugated to a BBB drug-targeting system, such as the OX26 antibody to the transferrin receptor in rats and the insulin receptor in humans. These antibodies accesses the endogenous transferrin transport system within the BBB and undergo receptor-mediated transcytosis through the intact BBB in vivo (Bickel et al., 1994). The time window of neuroprotection with the bFGF conjugate is 1 to 2 hours following a single intravenous injection of low doses (5-25 μg/kg) of the bFGF-TA conjugate (Table 3). This period is less than the 3-hour time window of neuroprotection following the constant intravenous infusion of high doses (135 μg/kg) of unconjugated bFGF (Ren and Finkelstein, 1997). The therapeutic time window for the bFGF conjugate may be prolonged either by increasing the dose or administering the bFGF-TA conjugate by constant intravenous infusion rather than a single intravenous bolus injection.

The neuroprotective effects of bFGF may be additive with other neurotrophins, such as brain-derived neurotrophic factor (BDNF), which is neuroprotective following direct intracerebral injection in regional brain ischemia (Yamashita et al., 1997). The BDNF must be given directly into the brain because it does not enter the brain following intravenous administration in the absence of BBB disruption (Sakane and Pardridge, 1997). The intravenous administration of unconjugated BDNF provides no neuroprotection in either global or regional brain ischemia (Wu and Pardridge, 1999; Zhang and Pardridge, 2001a,b). Conversely, the conjugate of BDNF and the OX26 antibody is neuroprotective following the delayed intravenous administration of low doses of the neurotrophin in either global or regional brain ischemia (Wu and Pardridge, 1999; Zhang and Pardridge, 2001a,b). BDNF is primarily neuroprotective in the cortex of the brain (Yamashita et al., 1997; Zhang and Pardridge, 2001b), whereas bFGF is neuroprotective in both cortical and subcortical regions of the brain (Fisher et al., 1995). Therefore, the combined use of bFGF and BDNF conjugates, which are enabled to cross the BBB may have additive effects as neuroprotective agents to brain ischemia. Dual neurotrophin therapy may also increase the therapeutic time window after the stroke during which neuroprotection is still possible.

TABLE 3
Neuroprotective effect of bFGF analogs on infarct volume
and neurological deficit Data are mean ± S.D.

| | | Neurological | |
|---|---|---|---|
| | Infarct | Deficit Score | |
| Treatment | Volume (mm³) | 2 h | 24 h |
| Vehicle (n-9) | 361 ± 29 | 2.6 ± 0.2 | 3.7 ± 0.4 |
| bio-bFGF/OX26-SA, 5 μg/kg | 238 ± 39** | 2.3 ± 0.2 | 3.2 ± 0.6 |
| (n-10) | | | |
| bio-bFGF/OX26-SA, 1-h delay, | 122 ± 36* | 2.1 ± 0.2 | 1.5 ± 0.2 |
| 25 μg/kg (n-10) | | | |
| bio-bFGF/OX26-SA, 2-h delay, | 358 ± 30 | 2.7 ± 0.3 | 3.0 ± 1.0 |
| 25 μg/kg (n-3) | | | |
| bio-bFGF/OX26-SA, 3-h delay, | 356 ± 32 | 2.7 ± 0.6 | 3.0 ± 1.0 |
| 25 μg/kg (n-3) | | | |

*p < 0.01;
**p < 0.05;
ANOVA with Bonferroni correction (F value − 12.4, degrees of freedom − 5).

Clinical trials have shown that bFGF produces dose-dependent hypotension in patients with ischemic heart disease (Laham et al., 2000) and leukocytosis in patients with acute ischemic stroke (Fiblast Safety Study Group, 1998). In the absence of a BBB drug-delivery system in accordance with the present invention, bFGF penetration into the brain is slow and occurs via an absorptive-mediated transcytosis mechanism (Deguchi et al., 2000), and this poor penetration of the BBB necessitates the administration of high systemic doses of bFGF when the neurotrophin is not reformulated to enable BBB transport (Fisher et al., 1995). The therapeutic effect of bFGF within the brain may be offset by the dose-dependent peripheral side effects caused by the administration of high doses by bFGF. The conjugation of bFGF to the present BBB drug-targeting system has dual beneficial effects. First, BBB transport of the bFGF is increased, which enables neuroprotection with bFGF conjugates at low systemic doses of 25 μg/kg (FIG. 2). Second, conjugation of bFGF to the BBB drug-delivery vector results in decreased peripheral organ distribution.

The size of the OX26-SA conjugate is 200,000 Daltons, and conjugation of bFGF to OX26-SA increases the effective molecular mass of the bFGF from 16,000 to 216,000 Daltons. The larger size of the conjugate restricts transcapillary transport into peripheral tissues, although the conjugate is selectively transported across cerebral capillaries. Therefore, the use of the present BBB drug-delivery system optimizes the therapeutic index of bFGF by simultaneously increasing central nervous system uptake and decreasing peptide uptake in peripheral tissues. This phenomenon has demonstrated previously with a vasoactive intestinal peptide analog, and conjugation of vasoactive intestinal peptide to OX26-SA increased the therapeutic index of the peptide 10-fold (Wu and Pardridge, 1996).

In summary, conjugation of bFGF to a BBB drug-delivery vector such as OX26-SA does not diminish the biological activity of the bFGF in a cell culture neuroprotection model (FIG. 5) or in a radio receptor assay (Wu et al., 2000). Neuroprotection is demonstrated in vivo with the permanent MCAO model, and a single intravenous administrative of the bFGF/OX26 conjugate results in an 80% reduction in stroke volume at a low systemic dose (25 μg/kg) of bFGF (FIG. 6). This dose of unconjugated bFGF has no significant effect on infarct volume following intravenous administration (FIG. 6). The in vivo neuroprotection of the bFGF/OX26 conjugate is dose-dependent and has an effective time window of at least 1 hour post-MCAO. Additional details regarding this example are set forth in Song et al., 2002.

### EXAMPLE 3

The OX26 antibody is specific for rats and would not be used in human applications. For humans, the most active BBB transport agent is the human insulin receptor (HIR) monoclonal antibody (MAb), or HIRMAb (Pardridge, 2001). The HIRMAb can be genetically engineered to form a mouse/human chimeric HIRMAb, and the activity of the chimeric HIRMAb is identical to the original mouse HIRMAb (Coloma et al, 2000). Humanized forms of the HIRMAb may also be used to target drugs across the human BBB. Exemplary, humanized monoclonal antibodies to the human insulin receptor that are particularly well-suited for use in the present invention are described in detail in copending application UC No. 2003-078-1 (Attorney Docket No. 0180-0038). A human patient suffering from cerebral ischemia (stroke) is treated with bio-bFGF conjugated to a fusion protein comprised of avidin or SA and the chimeric or humanized HIRMAb as described in detail in the previously referenced co-owned and co-pending United States patent application. The bio-bFGF/HIRMAb-SA or bio-bFGF/HIRMAb-avidin conjugate is prepared in the same manner as Example 1. The bFGF is biotinylated, the HIRMAb is conjugated with avidin or streptavidin and the two resulting compounds are combined to form the final conjugate. The conjugate is combined with a carrier solution of buffered water or saline and injected intravenously into the patient. The initial dose is 5-25 μg/kg. The lower dose may be administered if the patient is treated within 1-2 hours since the onset of cerebral ischemia. If a longer time has elapsed, then a higher dose should be administered. The actual dosages that give maximal drug effect in brain and minimal toxic effects in peripheral tissues will become apparent with continued use of the invention.

### EXAMPLE 4

The bFGF may be attached to the chimeric or humanized HIRMAb, not with avidin-biotin technology, but with genetic engineering that avoids the need for biotinylation or the use of foreign proteins such as SA or avidin. In this approach, the gene encoding for bFGF is fused to the region of the HIRMAb heavy chain or light chain gene corresponding to the amino or carboxyl terminus of the HIRMAb heavy or light chain protein. Following construction of the fusion gene and insertion into an appropriate prokaryotic or eukaryotic expression vector, the HIRMAb/bFGF fusion protein is mass produced for purification and manufacturing.

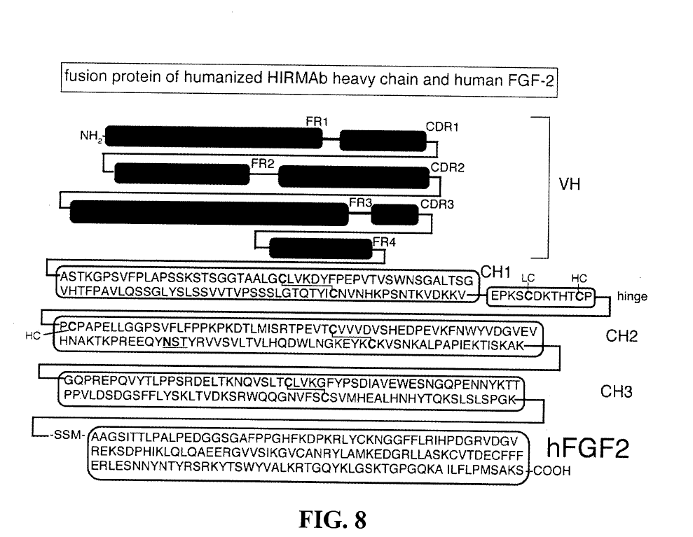

The amino acid sequence and general structure of a typical MAb/FGF2 fusion protein is shown in FIG. 8. The amino acid sequence for the FGF-2 shown in FIG. 8 is that of the 18 kDa variant of human FGF-2. It is well known that this cytokine is expressed as at least 4 different variants, called the 18 kDa, the 22 kDa, the 22.5 kDa, and the 24 kDa variants. The amino acid sequence of the 22 kDa, the 22.5 kDa, or the 24 kDa human FGF-2 variants could also be fused to the carboxyl terminus of the HIRMAb HC. Alternatively, any of the FGF-2 variants could be fused to the amino terminus of the HIRMAb HC or the amino or carboxyl termini of the HIRMAb light chain (LC). In addition, one or more amino acids within the FGF-2 sequence could be modified with retention of the biological activity of the FGF-2. The HIRMAb is a model blood-brain barrier targeting agent (TA) and could be substituted by other TAs such as insulin, transferrin, leptin, IGFs or a corresponding peptidomimetic MAb to the cognate receptors for these endogenous ligands. Biologically active fusion proteins of FGF-2 have been prepared and these fusion proteins retain biological activity. FGF2 has been fused to the amino terminus of saporin and the fusion protein has been expressed in bacteria (McDonald et al, 1996). FGF has been fused to the carboxyl terminus of human IgG and produced in bacteria (Dikov et al, 1998). FGF2 has been fused to the amino terminus of a recombinant lectin and expressed in bacteria (Schmidt et al, 2000). Human IgG/cytokine fusion proteins have been genetically engineered and expressed in eukaryotic myeloma expression systems (Penichet et al, 2000).

As is apparent form the preceding description, the present invention provides a substantial increase in the transport of biologically active bFGF across the BBB. In addition, the invention reduces the amount of bFGF that is taken up by other tissues and organs. This combination of increased BBB targeting and transport is especially useful since bFGF can now be injected intravenously in amounts that are below toxic levels while still providing effective neuroprotection for patients suffering from cerebral stroke.

Having thus described exemplary embodiments of the present invention, it should be noted by those skilled in the art that the within disclosures are exemplary only and that various other alternatives, adaptations and modifications may be made within the scope of the present invention. Accordingly, the present invention is not limited to the above preferred embodiments and examples, but is only limited by the following claims.

### BIBLIOGRAPHY

Abe, K. and Saito, H. (2000) “Neutrophic effect of basic fibroblasts growth factor is mediated by the p42/p44 mitogen-activates protein kinase cascade in cultured rat cortical neurons”, Develop. Brain Res. 122, 81-85.

Albayrak, S., Zhao, Q., Siesjo, B. K., and Smith, M-L (1997) “Effect of transient local ischemia on blood-brain barrier permeability in the rat correlation to cell injury,” Acta Neuropathol 94:158-163.

Ay, H., Ay, I., Koroshtz, W. J., and Finklestein, S. P. (1999) “Potential usefulness of basic fibroblast growth factor as a treatment for stroke,” Cerebrocase Dis 9:131-135.

Belayev, L., Busto, R., Zhao, W., and Ginsberg, M. D. (1996) “Quantitative evaluation of blood-brain barrier permeability following middle cerebral artery occlusion in rats, Brain Res 739:88-96.

Bickel, U., Kang, Y-S., Yoshikkawa, T., and Pardridge, W. M. (1994) “In vivo demonstration of subcellular localization of anti-transferrin receptor monoclonal antibody-colloidal gold conjugate within brain capillary endothelium,” J. Histochem Cytochem, 14:1493-1497.

Bikfalvi, A., Klein, S., Pintucci, G., and Rifkin, D. B. (1997) “Biological roles of fibroblasts growth factor-2,” Endocrine Rev. 18:26-45.

Brandoli, C., Sanna, A., De Bernardi, M. A., Follesa, P., Brooker, G. and Mochetti, I. (1998) “Brain-derived neurotrophic factor and basic fibroblast growth factor downregulate NMDA receptor function in granule cells,” J. Neurosci. 18:7953-7961.

Cambron, H., Latulippe, J-F., Nguyen, T. and Cartier, R. (1995) “Orotracheal intubation of rats by transillumination,” Lab Anum Sci 45:303-304.

Casper, D. and Blum, M. (1995) “Epidermal growth factor and basic fibroblast growth factor protect dopaminergic neurons from glutamate toxicity in clutures,” J. Neurochem. 65:1016-1026.

Cazevieille, C., Muller, A., Meynier, F., and Bonne, C. (1993) “Superoxide and nitric oxide cooperation in hypoxia/reoxygenation-induced neuron injury,” Free Radic Biol Med 14:389-395.

Clark, W. M., Schim, J. D., Kasner, S. E., and Victor, S. J. (2000) “Trafermin in acute ischemic stroke: results of a phase II/III randomized efficacy study,” Neurology 54 (Suppl. 3) A88.

Coloma, M. J., Lee, H. J., Kurihara, A., Landaw, E. M., Boado, R. J., Morrison, S. L., and Pardridge, W. M. (2000) “Transport across the primate blood-brain barrier of a genetically engineered chimeric monoclonal antibody to the human insulin receptor,” Pharm. Res. 17, 266-274.

Deguchi, Y., Kurihara, A., and Pardridge, W. M. (1999) “Retention of biologic activity of human epidermal growth factor following conjugation to a blood-brain barrier drug delivery vector via an extended polyethyleneglycol linker,” Bioconj. Chem., 10:32 -37.

Deguchi, Y., Naito, T., Yuga, T., Furukawa, A., Yamada, S., Pardridge, W. M. and Kimura, R. (2000) “Blood-brain barrier transport of ¹²⁵I-labeled fibroblast growth factor,” Pharm. Res. 17:63-69.

Dikov, M. M. et al (1998) A functional fibroblast growth factor-1 immunoglobulin fusion protein. J. Biol. Chem. 273: 15811-15817.

Dore, S., Kar, S. and Quinon, R. (1997) Insulin-like growth factor I protects and rescues hippocampal neurons against p-amyloid and human amyl in-induced toxicity. Proc. Natl. Acad. Sci. USA 94:4772-4777.

Fiblast Safety Study Group (1998) Clinical safety trial of intravenous basic fibroblast growth factor (bFGF, Fiblast) in acute stroke, Abstract, Stroke 29:287.

Fisher, M., Meadows, M.-E., Do, T., Weise, J., Trubetskoy, V., Charette, M. and Finklestein, S. P. (1995) “Delayed treatment with intravenous basic fibroblast growth factor reduces infarct size following permanent focal cerebral ischemia in rats,” J. Cereb. Blood Flow Metabol. 15:953-959.

Gospodarowicz, D. (1984) In: Pagel, W. J., ed. Mediators in Cell Growth and Differentiation (Raven Press, New York), pp. 109-134.

Guo, Q., Sebastian, L., Sopher, B. L., Miller, M. W., Glazner, G. W., Ware, C. B. and Mattson, M. P. (1999) “Neurotrophic factors [activity-dependent neurotrophic factor (ADNF) and basic fibroblast growth factor (bFGF)] interrupt excitotoxic neurodegenerative cascades promoted by a PSI mutation,” Proc. Natl. Acad. Sci. USA 96:4125-4130.

Hakan, A., Ikknur, A., Koroshtz, W. J., and Finklestein, S. P. (1999) “Potential usefulness of basic fibroblast growth factor as a treatment for stroke,” Cereb. Dis. 9:131-135.

Harukum, I., Traystman, R. J., Bhardwaj, A., Koehler, R. C., and Kirsch, J. R. (1998) “Intravenous basic fibroblast growth factor does not ameliorate brain injury resulting from transient focal ischemia in cats,” J. Neurosurg. Anesthsiol 10:160-165.

Jefferies, W. A., Brandon, M. R., Hunt, S. V., Williams, A. F., Gattters, K. C., and Mason, D. Y. (1984) Transferrin receptor on endothelium of brain capillaries. Nature 312, 162-163.

Jiang, N., Finklestein, S. P., Do, T., Caday, C. G., Charette, M. and Choop, M. (1996) “Delayed intravenous administration of basic fibroblast growth (bFGF) reduces infarct volume in a model of focal cerebral ischemia/reperfusion in the rat,” J. Neurol. Sci. 139:173-179.

Jostock, T. et al. (1999) “Immunoadhesins of interleukin-6 and the IL-6/soluble IL-6R fusion protein hyper-IL-6,” J. Immunol. Meth., 223: 171-183.

Kang, Y.-S., and Pardridge, W. M. (1994) “Use of neutral-avidin improves pharmacokinetics and brain delivery of biotin bound to an avidin monoclonal antibody conjugate,” J. Pharmacol. Exp. Ther. 269:344-350.

Kawai, N., Keep, R. F., and Betz, A. L. (1997) “Hyperglycemia and the vascular effects of cerebral ischemia,” Stroke, 28:149-154.

Kiyokawa, N., Lee, E. K., Karunagaran, D., Lin, S.-Y., and Hung, M.-C. (1997) “Mitosis-specific negative regulation of epidermal growth factor receptor, triggered by a decrease in ligand binding and dimerization, can be overcome by overexpression of receptor,” J. Biol. Chem. 272:18656-18665.

Koketsu, N., Berlove, D. J., Moskowitz, M. A., Kowall, N. W., Caday, C. G. and Finklestein, S. P. (1994) “Pretreatment with intraventricular basic fibroblast growth factor (bFGF) decreases infarct size following focal cerebral ischemia in rats,” Ann. Neurol. 35:451-457.

Lahman, R. J., Chronos, N. A., Pikes, M., Leimbach, M. E., Udelson, J. E., Pearlman, J. D., Pettigrew, R. I., Whitehouse, M. J., Yoshiizawa, C. and Simons, M. (2000) “Intracoronary basic fibroblast growth factor (FGF-2) in patients with severe ischemia heart disease: results of a Phase-I open-labeled close escalation study,” J. Am. College Cardiol. 36:2132-2139.

Lee, H. J., Engelhardt, B., Lesley, J., Bickel, U., and Pardridge, W. M. (2000) Targeting rat anti-mouse transferrin receptor monoclonal antibodies through the blood-brain barrier in the mouse. J. Pharmacol. Exp. Ther. 292, 1048-1052.

Liu, X., Zhu, X.-Z., and Ji, X.-Q. (1999) “Effect of basic fibroblast growth factor on focal ischemic injury and antioxidant enzyme activities,” Acta Pharmacol. Sin 20:277-331.

Luo, J., West, J. R. and Pantazis, N. J. (1997) “Nerve growth factor and basic fibroblast growth factor protect rat cerebellar granule cells in culture against ethanol-induced cell death,” Alchol. Clin. Exp. Res. 21:1108-1120.

Lyons, M. K., Anderson, R. E., and Meyer, F. B. (1991) “Basic fibroblast growth factor promotes in vivo cerebral angiogenesis in chronic forebrain ischemia,” Brain Res. 558:315-320.

Matton, M. P. and Barger, S. W. (1995) “Programmed cell life: Neuroprotective signal transduction and ischemic brain injury,” In: Moskowitz, M. A. and Caplan, L. R. eds., Cerebrovascular Disease 19th Princeton Stroke Conference. (Butterworth-Heinemann, Newton), pp. 271-290.

Mazue, G., Bertolero, F., Garaofano, L., Brughera, M. and Cariminati, P. (1992) “Experience with the preclinical assessment of basic fibroblast growth factor (bFGF),” Toxicol. Lett. 64-65:329-338.

Mazue, G., Newman, A. J., Scampini, G., Della Torre, P. and Dagnasco, S. M. (1993) “The histopathology of kidney changes in rats and monkeys following intravenous administration of massive doses of FCE 2184, human basic fibroblast growth factor,” Toxicol. Pathol. 21:490-501.

McDonald, J. R. et al (1996) Large-scale purification and characterization of recombinant fibroblast growth factor-saporin mitotoxin. Prot. Exp. Purif. 8, 97-108.

Menzies, S. A., Betz, A. L., and Hoff, J. T. (1993) “Contributions of ions and albumin to the formation and resolution of ischemic brain edema,” J. Neurosurg. 78:257-266.

Nakagami, Y., Saito, H. and Matsuki, N. (1997) “Basic fibroblast growth factor and brain-derived neutrotrophic factor promote survival and neuronal circuit,” Jpn J. Pharmacol.75:319-326.

Neufeld, G. and Gospodarowicz, D. (1985) “The identification and partial characterization of the fibroblast growth factor receptor of baby hamster kidney cells,” J. Biol. Chem. 26:13860-13868.

Neufeld, G. and Gospodarowiicz, D. (1986) “Basic and acidic fibroblast growth factors interact with the same cell surface receptors,” J. Biol. Chem. 261:5631-5637.

Pardridge, W. M. (2001) Brain Drug Targeting, Cambridge University Press, Cambridge, UK.

Pardridge, W. M., Buciak, J. L., and Friden, P. M. (1991) “Selective transport of anti-transferrin receptor antibody through the blood-brain barrier in vivo,” J. Pharmacol. Exp. Ther. 256:66-70.

Pardridge, W. M., Kang, Y. S. and Buctak, J. L. (1994) “Transport of human recombinant brain derived neutrophic factor (BDNF) through the rat blood-brain barrier in vivo using vector-mediated peptide drug delivery,” Pharm. Res. 11:738-746.

Penichet, M. L. et al. (2002) “A recombinant IgG3-(IL-2) fusion protein for the treatment of human HER2/neu expressing tumors,” Human Antibodies, 10:43-49.

Ren, J. M. and Finklestein, S. P. (1997) “Time window of infarct reduction by intravenous basic fibroblast growth factor in local cerebral ischemia,” Eur. J Pharmacol. 327:11-16.

Roberts, T. P., Vexler, Z. S., Denrugin, N., Kozmewska, E., Kucharczyk, J. and Emmet, C. J. (1995) “Evaluation of recombinant human basic fibroblast growth factor (rhbFGF) as a cerebroprotective agent using high speed MR imaging,” Brain Res. 699:51-61.

Roghani, M. and Moscatelli, D. (1992) “Basic fibroblast growth factor is internalized through both receptor-mediated and heparan sultate-mediated mechanisms,” J. Biol. Chem. 267:22156-22162.

Sakane, T. and Pardridge, W. M. (1997) “Carboxyl-directed pegylation of brain-derived neurotrophic factor markedly reduces systemic clearance with minimal loss of biologic activity,” Pharm. Res. (NY) 14:1085-1091.

Schmidt, A. et al. (2000) “Cytotoxic activity of recombinant bFGF-rViscumin fusion proteins,” Biochem. Biophys. Res. Comm., 277:499-506.

Song, B.-W., Vinters, H. V., Wu, D. and Pardridge, W. M. (2002) “Enhanced Neuroprotective Effects of Basic Fibroblast Growth Factor in Regional Brain Ischemia after Conjugation to a Blood-Brain Barrier Delivery Vector,” JPET 301:605-610.

Tatlisumak, T., Takano, K., Carano, R. A. and Fisher, M. (1996) “Effects of basic fibroblast growth factor on experimental focal ischemia studied by diffusion-weighted and perfusion imaging,” Stroke 27:2292-2297.

Triguero, D., Buctak, J. and Pardridge, W. M. (1990) “Capillary depletion method quantification of blood-brain barrier transport of circulating peptides and plasma proteins,” J. Neurochem. 54:1882-1888.

Whalen, G. F., Shing, Y. and Folkman, J. (1989) “The fate of intravenously administered bFGF and the effect of heparin,” Growth Factors 1:157-164.

Wu, D. and Pardridge, W. M. (1996) “Central nervous system pharmacologic effect in conscious rats after intravenous injection of a biotinylated vasoactive intestinal peptide analog coupled to a blood-brain barrier drug delivery system,” J. Pharmacol. Exper. Ther. 279:77-83.

Wu, D. and Pardridge, W. M. (1999) “Neuroprotection with non-invasive neurotrophin delivery to brain,” Proc. Natl. Acad. Sci. USA 96:251-259.

Wu, D., Song, B.-W., Vinters, H. V., and Pardridge, W. M. (2002) “Pharmacokinetics and brain uptake of biotinylated basic fibroblast growth factor conjugated to a blood-brain barrier drug delivery system,” J. Drug Target 10:239-246.

Wu, D., Boado, R. J. and Pardridge, W. M. (1996) “Pharmacokinetics and blood-brain barrier transport of [³H]-biotinylated phosphorothioate oligodeoxynucleotide conjugated to a vector-mediated drug delivery system,” J. Pharm. Exp. Ther. 276:206-211.

Yamada, K., Kinoshita, A., Koshmora, E., Sakaguchi, T., Tuguchi, J., Kataoka, K. and Hayakawa, T. (1991) “Basic fibroblast growth factor prevents thalamic degeneration after cortical infarction,” J. Cereb. Blood Floow Metabol. 11:472-478.

Yamashita, K., Wiessner, C., Lindholm, D., Thoenen, H., and Hossthann, K.-A. (1997) “Post-occlusion treatment with BDNF reduces infarct size in a model of permanent occlusion of the middle cerebral artery in rat,” Metab. Brain. Dis. 12:271-281.

Zhang, Y. and Pardridge, W. M. (2001a) “Conjugation of brain-derived neurotrophic factor to a blood-brain barrier drug targeting system enables neuroprotection in regional brain ischemia following intravenous injection of the neurotrophin,” Brain Res. 889:49-56.

Zhang, Y. and Pardridge, W. M. (2001b) “Neuroprotection in transient focal brain ischemia following delayed, intravenous administration of BDNF conjugated to a blood-brain barrier drug targeting system,” Stroke 32:1378-1384.

What is claimed is:

1. A composition that is capable of undergoing receptor mediated transport across the blood brain barrier, said composition comprising:
a biotinylated basic fibroblast growth factor comprising basic fibroblast growth factor and biotin; and
a transport vehicle comprising a blood brain barrier targeting agent and avidin or streptavidin, said transport vehicle having at least one biotin binding site and being capable of undergoing receptor mediated transport across the blood brain barrier, wherein said transport vehicle is conjugated to said biotinylated basic fibroblast growth factor to form said composition that is capable of undergoing receptor mediated transport across the blood brain barrier, said transport vehicle being conjugated to said biotinylated basic fibroblast growth factor by a bond between said biotin and said avidin or streptavidin at said biotin binding site.

2. A composition according to claim 1 wherein said avidin or streptavidin has one to four biotin binding sites.

3. A composition according to claim 1 wherein said targeting agent is selected from the group consisting of insulin, transferrin, insulin-like growth factor (IGF), leptin, low density lipoprotein (LDL), and monoclonal antibodies that bind to the insulin, IGF, leptin or LDL receptor on the blood brain barrier.

4. A composition according to claim 3 wherein said targeting agent is a monoclonal antibody to the human insulin receptor.

5. A composition according to claim 1 wherein said biotinylated basic fibroblast growth factor is monobiotinylated.

6. A pharmaceutical composition that comprises a composition according to claim 1 and a pharmaceutically acceptable carrier for said composition.

7. A pharmaceutical composition according to claim 6 wherein said avidin or streptavidin has one to four biotin binding sites.

8. A pharmaceutical composition according to claim 6 wherein said targeting agent is selected from the group consisting of insulin, transferrin, insulin-like growth factor (IGF), leptin, low density lipoprotein (LDL), and monoclonal antibodies that bind to the insulin, IGF, leptin or LDL receptor on the blood brain barrier.

9. A pharmaceutical composition according to claim 8 wherein said targeting agent is a monoclonal antibody to the human insulin receptor.

10. A pharmaceutical composition according to claim 6 wherein said biotinylated basic fibroblast growth factor is monobiotinylated.

11. A method for increasing the transportability of a basic fibroblast growth factor across the blood brain barrier, said method comprising the steps of:
conjugating said basic fibroblast growth factor with a blood brain barrier targeting agent wherein said conjugating step comprises reacting biotinylated basic fibroblast growth factor with a transport vehicle comprising said blood brain barrier targeting agent and avidin or streptavidin, said biotinylated basic fibroblast growth factor comprising basic fibroblast growth factor and biotin, said transport vehicle having at least one biotin binding site and being capable of undergoing receptor mediated transport across the blood brain barrier, said transport vehicle being conjugated to said biotinylated basic fibroblast growth factor by a bond between said biotin and said avidin or streptavidin at said biotin binding site.

12. A method according to claim 11 wherein said transport vehicle has three biotin binding sites.

13. A method according to claim 11 wherein said targeting agent is selected from the group consisting of insulin, transferrin, insulin-like growth factor (IGF), leptin, low density lipoprotein (LDL), and monoclonal antibodies that bind to the insulin, IGF, leptin or LDL receptor on the blood brain barrier.

14. A method according to claim 13 wherein said targeting agent is a monoclonal antibody to the human insulin receptor.

15. A method according to claim 11 wherein said biotinylated basic fibroblast growth factor is monobiotinylated.

16. A method for delivering basic fibroblast growth factor across the blood brain barrier, said method comprising the step of intravenously administering to an animal a preparation comprising:
A) a composition that is capable of undergoing receptor mediated transport across the blood brain barrier, said composition comprising:
a) a biotinylated basic fibroblast growth factor comprising basic fibroblast growth factor and biotin;
b) a transport vehicle comprising a blood brain barrier targeting agent and avidin or streptavidin, said transport vehicle having at least one biotin binding site and being capable of undergoing receptor mediated transport across the blood brain barrier, wherein said transport vehicle is conjugated to said biotinylated basic fibroblast growth factor to form said composition that is capable of undergoing receptor mediated transport across the blood brain barrier, said transport vehicle being conjugated to said biotinylated basic fibroblast growth factor by a bond between said biotin and said avidin or streptavidin at said biotin binding site; and
B) a pharmaceutically acceptable carrier for said composition.

17. A method according to claim 16 wherein said transport vehicle has up to four biotin binding sites.

18. A method according to claim 16 wherein said targeting agent is selected from the group consisting of insulin, transferrin, insulin-like growth factor (IGF), leptin, low density lipoprotein (LDL), and monoclonal antibodies that bind to the insulin, IGF, leptin or LDL receptor on the blood brain barrier.

19. A method according to claim 18 wherein said targeting agent is a monoclonal antibody to the human insulin receptor.

20. A method according to claim 16 wherein said biotinylated basic fibroblast growth factor is monobiotinylated.

21. A method for reducing the size of a cerebral infarction in an animal, said method comprising the step of intravenously administering to said animal an amount of a preparation that is sufficient to reduce the size of said cerebral infarction, said preparation comprising:
A) a composition that is capable of undergoing receptor mediated transport across the blood brain barrier, said composition comprising:
a) a biotinylated basic fibroblast growth factor comprising basic fibroblast growth factor and biotin;
b) a transport vehicle comprising a blood brain barrier targeting agent and avidin or streptavidin, said transport vehicle having at least one biotin binding site and being capable of undergoing receptor mediated transport across the blood brain barrier, wherein said transport vehicle is conjugated to said biotinylated basic fibroblast growth factor to form said composition that is capable of undergoing receptor mediated transport across the blood brain barrier, said transport vehicle being conjugated to said biotinylated basic fibroblast growth factor by a bond between said biotin and said avidin or streptavidin at said biotin binding site; and
B) a pharmaceutically acceptable carrier for said composition.

22. A method according to claim 21 wherein the amount of said preparation that is administered intravenously to said animal is sufficient to provide a dose of basic fibroblast growth factor of between about 1 μg and 50 μg per kilogram of animal body weight.

23. A method according to claim 21 wherein said transport vehicle has up to four biotin binding sites.

24. A method according to claim 21 wherein said targeting agent is selected from the group consisting of insulin, transferrin, insulin-like growth factor (IGF), leptin, low density lipoprotein (LDL), and monoclonal antibodies that bind to the insulin, IGF, leptin or LDL receptor on the blood brain barrier.

25. A method according to claim 24 wherein said targeting agent is a monoclonal antibody to the human insulin receptor.

26. A method according to claim 25 wherein said animal is a human being.

27. A method according to claim 21 wherein said biotinylated basic fibroblast growth factor is monobiotinylated.

28. A fusion protein that is capable of undergoing receptor mediated transport across the blood brain barrier, said fusion protein comprising basic fibroblast growth factor and a blood brain barrier targeting agent that is capable of undergoing receptor mediated transport across the blood brain barrier, said blood brain barrier targeting agent being fused to said basic fibroblast growth factor.

29. A fusion protein according to claim 28 wherein said targeting agent is selected from the group consisting of insulin, transferrin, insulin-like growth factor (IGF), leptin, low density lipoprotein (LDL), and monoclonal antibodies that bind to the insulin, IGF, leptin or LDL receptor on the blood brain barrier.

30. A fusion protein according to claim 29 wherein said targeting agent is a monoclonal antibody that binds to the human insulin receptor.

31. A method for increasing the transportability of a basic fibroblast growth factor across the blood brain barrier, said method comprising the step of fusing said basic fibroblast growth factor with a blood brain barrier targeting agent to provide a fusion protein that is capable of being transported across the blood brain barrier.

32. A method according to claim 31 wherein said targeting agent is selected from the group consisting of insulin, transferrin, insulin-like growth factor (IGF), leptin, low density lipoprotein (LDL), and monoclonal antibodies that bind to the insulin, IGF, leptin or LDL receptor on the blood brain barrier.

33. A method according to claim 32 wherein said targeting agent is a monoclonal antibody that binds to the human insulin receptor.

34. A method for delivering basic fibroblast growth factor across the blood brain barrier, said method comprising the step of intravenously administering to an animal a preparation comprising:
A) a fusion protein comprising basic fibroblast growth factor and a blood brain barrier targeting agent that is capable of undergoing receptor mediated transport across the blood brain barrier, said blood brain barrier targeting agent being fused to said basic fibroblast growth factor; and
B) a pharmaceutically acceptable carrier for said fusion protein.

35. A method according to claim 34 wherein said targeting agent is selected from the group consisting of insulin, transferrin, insulin-like growth factor (IGF), leptin, low density lipoprotein (LDL), and monoclonal antibodies that bind to the insulin, IGF, leptin or LDL receptor on the blood brain barrier.

36. A method according to claim 35 wherein said targeting agent is a monoclonal antibody that binds to the human insulin receptor.

37. A method for reducing the size of a cerebral infarction in an animal, said method comprising the step of intravenously administering to said animal an amount of a preparation that is sufficient to reduce the size of said cerebral infarction, said preparation comprising:
A) a fusion protein comprising basic fibroblast growth factor and a blood brain barrier targeting agent that is capable of undergoing receptor mediated transport across the blood brain barrier, said blood brain barrier targeting agent being fused to said basic fibroblast growth factor; and
B) a pharmaceutically acceptable carrier for said fusion protein.

38. A method according to claim 37 wherein said targeting agent is selected from the group consisting of insulin, transferrin, insulin-like growth factor (IGF), leptin, low density lipoprotein (LDL), and monoclonal antibodies that bind to the insulin, IGF, leptin or LDL receptor on the blood brain barrier.

39. A method according to claim 38 wherein said targeting agent is a monoclonal antibody that binds to the human insulin receptor.
